(19) United States
(12) Patent Application Publication
(10) Pub. No.: US 2020/0157144 A1
(43) Pub. Date: May 21, 2020

(54) ANTIMICROBIAL PEPTIDOMIMETICS

(71) Applicant: Agency for Science, Technology and Research, Singapore (SG)

(72) Inventor: Brian Cheng San Chia, Singapore (SG)

(21) Appl. No.: 16/635,401

(22) PCT Filed: Jul. 2, 2018
(86) PCT No.: PCT/SG2018/050320

§ 371 Date: Jan. 30, 2020

(30) Foreign Application Priority Data
Aug. 1, 2017 (SG) 10201706251Q

(51) Int. Cl. C07K 5/08; C07K 5/10; C07K 5/06; C07K 7/06; A61K 9/00; A61P 31/04

(52) U.S. Cl. CPC C07K 5/08; C07K 5/10; C07K 5/06; A61K 38/00; A61K 9/0014; A61P 31/04; C07K 7/06

(57) ABSTRACT

The present invention relates to peptides containing guanidine and halogenated biphenyl moieties of the formula (I), and to salts and solvates of each of these peptides and to processes for the preparation thereof, compositions containing them and the uses of such compounds. It has been found that the compounds have a high microbicide activity and are suited to combat resistant bacteria, such as Methicillin-resistant Staphylococcus aureus (MRSA) strains, at very low concentrations.

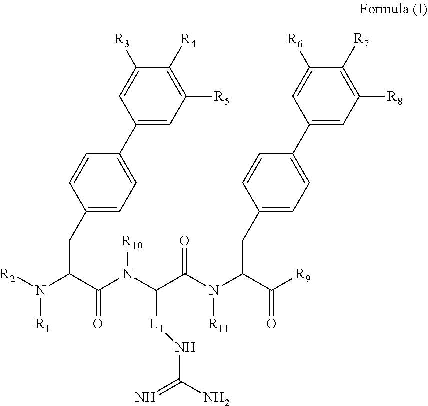

### TECHNICAL FIELD

The present invention relates to compounds, compositions, and methods for treating diseases and conditions. In particular, the invention relates to compounds, compositions, and methods for treating bacterial infections, disorders and conditions.

### BACKGROUND

In 2011, there were 80,000 cases of invasive Meticillin-resistant Staphylococcus aureus (MRSA) infections in the United States, resulting in 11,000 fatalities. The emergence of multi-drug-resistant bacteria and the lack of new antibiotics in the drug development pipelines of pharmaceutical companies is a major health concern. Since 2000, only four antibiotics with new chemical scaffolds were launched; the (i) oxazolidinone Linezolid (2000), (ii) lipopeptide Daptomycin (2003), (iii) pleuromutilin Retapamulin (2007) and (iv) macrocycle Fidaxomicin (2011). Hence, there is an urgent need to develop new classes of antibacterials, especially those against emerging multi-drug-resistant bacteria. Staphylococcus aureus (SA) is also the primary culprit responsible for human skin and soft tissue infections (SSTIs). A study involving 11 US hospitals revealed more than 75% of SSTI cases were caused by Staphylococcus aureus and close to 60% were found to involve Meticillin-resistant Staphylococcus aureus (MRSA). Of particular concern is the emergence of Mupirocin-resistant MRSA, a front-line widely-used topical antibiotic used for the treatment of MRSA skin infections and nasal decolonization. At Singapore's Tan Tock Seng hospital, the prevalence of Mupirocin-resistant MRSA was 11% in a 2009-2010 survey. Alarmingly, a more recent 2013 survey by 7 public-sector hospitals in Singapore revealed a 31% incidence rate, mirroring a 2012-2013 survey at a New York City hospital. The highest rate of Mupirocin resistance ever reported was 79% in 2008 in a Swiss hospital. With these alarming statistics, it is imperative that a Mupirocin substitute be developed as soon as possible.

### SUMMARY

Novel compounds have now been found with superior anti-bactericidal activities.

In one aspect, there is provided a compound of formula (I):

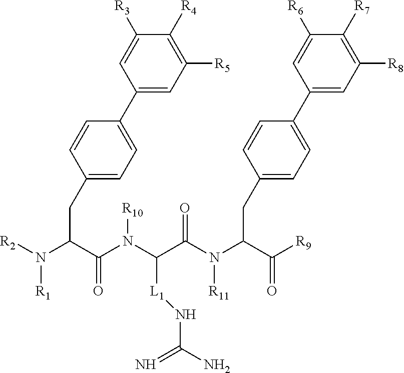

or a pharmaceutically acceptable salt or solvate thereof;
wherein L₁ is —(CH₂)ₘ—, C₂-C₅ alkenylene, C₂-C₅ alkynylene, C₃-C₆ cycloalkylene, phenylene or benzylene, wherein m is selected from 1 to 5;
wherein R₁ and R₂ are independently selected from H or CH₃, or

R₁ is selected from H or CH₃ and R₂ is

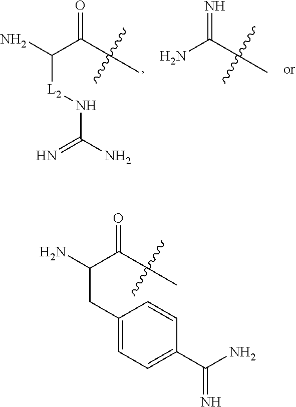

wherein L₂ is —(CH₂)ₙ—, C₂-C₅ alkenylene, C₂-C₅ alkynylene, C₃-C₆ cycloalkylene, phenylene or benzylene, wherein n is selected from 1 to 5;
wherein R₃-R₈ are independently H, Cl, I, Br or F;
wherein at least one of R₃-R₈ is Cl, I, Br or F;
wherein R₉

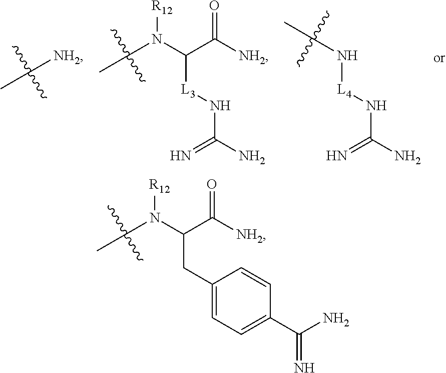

wherein L₃ is —(CH₂)ₒ—, C₂-C₅ alkenylene, C₂-C₅ alkynylene, C₃-C₆ cycloalkylene, phenylene or benzylene, wherein o is selected from 1 to 5;
wherein L₄ is —(CH₂)ₚ—, C₂-C₅ alkenylene, C₂-C₅ alkynylene, C₃-C₆ cycloalkylene, phenylene or benzylene, wherein p is selected from 1 to 5;
wherein R₁₀, R₁₁ and R₁₂ are independently H or —CH₃.

### BRIEF DESCRIPTION OF THE FIGURES

FIG. 1 shows a time-kill assay using compounds at 4×MIC concentration on Mupirocin-resistant MRSA (ATCC-BAA-1556).

FIG. 2 shows a bactericidal/static determination assay at 4×MIC concentration using Mupirocin-resistant MRSA (ATCC-BAA-1556); (A) Linezolid; (B) Retapamulin; (C) Vancomycin; (D) Compound 34.

FIG. 3 shows the minimum bactericidal concentration determination of Compound 34 using Mupirocin-resistant MRSA (ATCC-BAA-1556); (A) at MIC; (B) at 2×MIC.

FIG. 4 shows an electrospray ionization-mass spectrum (ESI-MS) of Compound 34.

FIG. 5 shows a nuclear magnetic resonance (NMR) spectrum of Compound 34.

### DETAILED DESCRIPTION OF THE INVENTION

### Definitions

The following are some definitions that may be helpful in understanding the description of the present invention. These are intended as general definitions and should in no way limit the scope of the present invention to those terms alone, but are put forth for a better understanding of the following description.

Unless the context requires otherwise or specifically stated to the contrary, integers, steps, or elements of the invention recited herein as singular integers, steps or elements clearly encompass both singular and plural forms of the recited integers, steps or elements.

Throughout this specification, unless the context requires otherwise, the word “comprise”, or variations such as “comprises” or “comprising”, will be understood to imply the inclusion of a stated step or element or integer or group of steps or elements or integers, but not the exclusion of any other step or element or integer or group of elements or integers. Thus, in the context of this specification, the term “comprising” means “including principally, but not necessarily solely”.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

The term “cycloalkyl” as used herein refers to cyclic saturated aliphatic groups and includes within its meaning monovalent (“cycloalkyl”), and divalent (“cycloalkylene”), saturated, monocyclic, bicyclic, polycyclic or fused polycyclic hydrocarbon radicals having from 3 to 10 carbon atoms, e.g., 3, 4, 5, 6, 7, 8, 9, or 10 carbon atoms. Examples of monovalent cycloalkyl groups include but are not limited to cyclopropyl, 2-methylcyclopropyl, cyclobutyl, cyclopentyl, 2-methylcyclopentyl, 3-methylcyclopentyl, cyclohexyl, and the like. Examples of divalent cycloalkylene groups include but are not limited to cyclopropylene, 2-methylcyclopropylene, cyclobutylene, cyclopentylene, 2-methylcyclopentylene, 3-methylcyclopentylene, cyclohexylene, and the like.

“Alkenylene” refers to divalent alkenyl groups preferably having from 2 to 8 carbon atoms and more preferably 2 to 6 carbon atoms. Examples include ethenylene (—CH═CH—), and the propenylene isomers (e.g., —CH₂CH═CH— and —C(CH₃)═CH—), and the like.

“Alkynylene” refers to the divalent alkynyl groups preferably having from 2 to 8 carbon atoms and more preferably 2 to 6 carbon atoms. Examples include ethynylene (—C≡C—), propynylene (—CH₂—C≡C—), and the like.

The term “phenylene” as used herein refers to a divalent benzene moiety.

The term “benzylene” as used herein refers to a divalent benzyl moiety of the following formula:

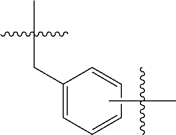

and preferably

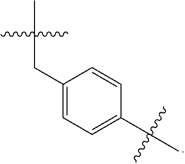 [SMILES: *Cc1ccc(*)cc1]

The present invention includes within its scope all isomeric forms of the compounds disclosed herein, including all diastereomeric isomers, racemates and enantiomers, unless the stereochemistry is fixed in the formula drawing. Thus, formula (I) should be understood to include, for example, E, Z, cis, trans, (R), (S), (L), (D), (+), and/or (−) forms of the compounds, as appropriate in each case, unless the stereochemistry is fixed in the formula drawing.

The term “Fmoc” or “fmoc” in the formulas and description refers to a typical fluorenylmethyloxycarbonyl protecting group.

The term “t-Boc” or “Boc” in the formulas and description refers to a typical tert-butoxycarbonyl protecting group.

The term “Pbf” stands for a 2,2,4,6,7-pentamethyldihydrobenzofuran-5-sulfonyl protecting group.

The term “a compound of formula (I) or a salt or solvate thereof” or “a compound of formula (I) or a pharmaceutically acceptable salt or solvate thereof” is intended to identify a compound selected from the group consisting of: a compound of the formula (I), a salt of a compound of formula (I), a pharmaceutically acceptable solvate of a compound of formula (I) or a pharmaceutically acceptable solvate of a pharmaceutically acceptable salt of a compound of formula (I).

The term “therapeutically effective” is intended to qualify the amount of compound or pharmaceutical composition, or the combined amount of active ingredients in the case of combination therapy.

The term “treatment” as used herein to describe the present invention und unless otherwise qualified, means administration of the compound, pharmaceutical composition or combination to effect preventive, palliative, supportive, restorative or curative treatment.

The term “preventive treatment” as used herein to describe the present invention, means that the compound, pharmaceutical composition or combination is administered to a subject or member of a population that is significantly predisposed to the relevant condition.

The term “palliative treatment” as used herein to describe the present invention, means that the compound, pharmaceutical composition or combination is administered to a subject to remedy signs and/or symptoms of a condition, without necessarily modifying the progression of, or underlying etiology of, the relevant condition. Non-limiting examples include reduction of pain, discomfort, swelling or fever.

The term “supportive treatment” as used herein to describe the present invention, means that the compound, pharmaceutical composition or combination is administered to a subject as part of a regimen of therapy, but that such therapy is not limited to administration of the compound, pharmaceutical composition or combination.

The term “restorative treatment” as used herein to describe the present invention, means that the compound, pharmaceutical composition or combination is administered to a subject to modify the underlying progression or etiology of a condition.

The term “preventive treatment” as used herein to describe the present invention, means that the compound, pharmaceutical composition or combination is administered to a subject for the purpose of bringing the disease or disorder into complete remission, or that the disorder is undetectable after such treatment.

The term “MIC” as used herein, means the minimum inhibitory concentration (MIC) is the lowest concentration of an antimicrobial that will inhibit the visible growth of a microorganism after overnight incubation.

The term “compounds of the invention” or “a compound of the invention” as used herein unless otherwise specified, means a compound of formula (I) or a pharmaceutically acceptable salt or solvate thereof.

Non-limiting examples of the above compounds according to the first aspect will now be disclosed.

In one aspect, there is provided a compound of formula (I):

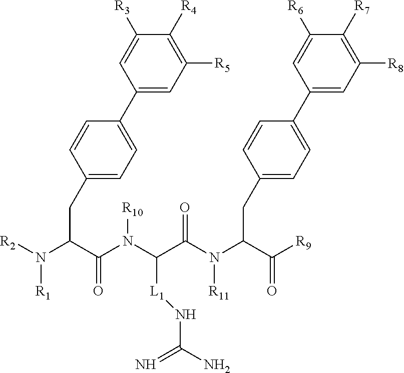

or a pharmaceutically acceptable salt or solvate thereof;
wherein L₁ is —(CH₂)ₘ—, C₂-C₅ alkenylene, C₂-C₅ alkynylene, C₃-C₆ cycloalkylene, phenylene or benzylene, wherein m is selected from 1 to 5;
wherein R₁ and R₂ are independently selected from H or CH₃, or R₁ is selected from H or CH₃, and R₂ is

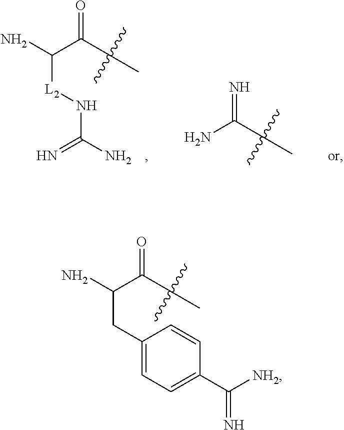

wherein L₂ is —(CH₂)ₙ—, C₂-C₅ alkenylene, C₂-C₅ alkynylene, C₃-C₆ cycloalkylene, phenylene or benzylene, wherein n is selected from 1 to 5;
wherein R₃-R₈ are independently H, Cl, I, Br or F;
wherein at least one of R₃-R₈ is Cl, I, Br or F;
wherein R₉ is

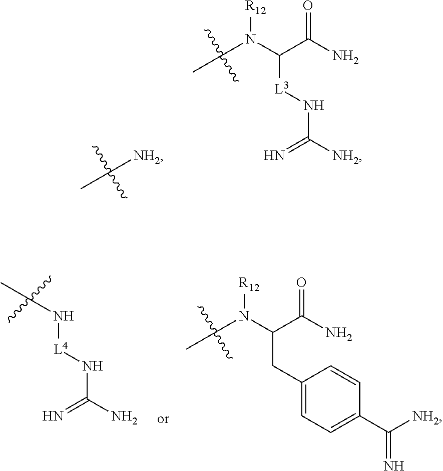

wherein L₃ is —(CH₂)ₒ—, C₂-C₅ alkenylene, C₂-C₅ alkynylene, C₃-C₆ cycloalkylene, phenylene or benzylene, wherein o is selected from 1 to 5;
wherein L₄ is —(CH₂)ₚ—, C₂-C₅ alkenylene, C₂-C₅ alkynylene, C₃-C₆ cycloalkylene, phenylene or benzylene, wherein p is selected from 1 to 5;
wherein R₁₀, R₁₁ and R₁₂ are independently H or —CH₃.

In an embodiment, L₁ is selected from —(CH₂)ₘ—, C₂-C₅ alkenylene, C₂-C₅ alkynylene, C₃-C₆ cycloalkylene, phenylene or benzylene, wherein m is selected from 1 to 5. In another embodiment, L₁ is —(CH₂)—. In another embodiment, L₁ is —(CH₂CH₂)—. In another embodiment, L₁ is —(CH₂CH₂CH₂)—. In another embodiment, L₁ is —(CH₂CH₂CH₂CH₂)—. In another embodiment, L₁ is —(CH₂CH₂CH₂CH₂CH₂)—. In another embodiment, L₁ is —(CH═CH)—. In another embodiment, L₁ is —(CH═CHCH₂)—. In another embodiment, L₁ is —(CH₂CH═CH)—. In another embodiment, L₁ is —(CH═CHCH₂CH₂)—. In another embodiment, L₁ is —(CH₂CH═CHCH₂)—. In another embodiment, L₁ is —(CH₂CH₂CH═CH)—. In another embodiment, L₁ is —(CH═CHCH₂CH₂CH₂)—. In another embodiment, L₁ is —(CH₂CH═CHCH₂CH₂)—. In another embodiment, L₁ is —(CH₂CH₂CH═CHCH₂)—. In another embodiment, L₁ is —(CH₂CH₂CH₂CH═CH)—. In another embodiment, L₁ is —(C≡C)—. In another embodiment, L₁ is —(C≡CCH₂)—. In another embodiment, L₁ is —(CH₂C≡C)—. In another embodiment, L₁ is —(C≡CCH₂CH₂)—. In another embodiment, L₁ is —(CH₂CCCH₂)—. In another embodiment, L₁ is —(CH₂CH₂C≡C)—. In another embodiment, L₁ is —(C≡CCH₂CH₂CH₂)—. In another embodiment, L₁ is —(CH₂CCCH₂CH₂)—. In another embodiment, L₁ is —(CH₂CH₂CCCH₂)—. In another embodiment, L₁ is —(CH₂CH₂CH₂C≡C)—.

In one embodiment, L₁ is selected from

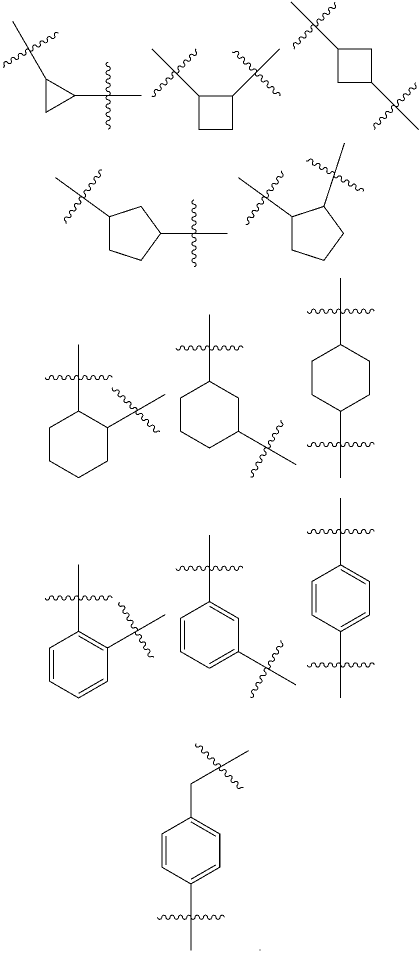

In another embodiment, L₁ is selected from

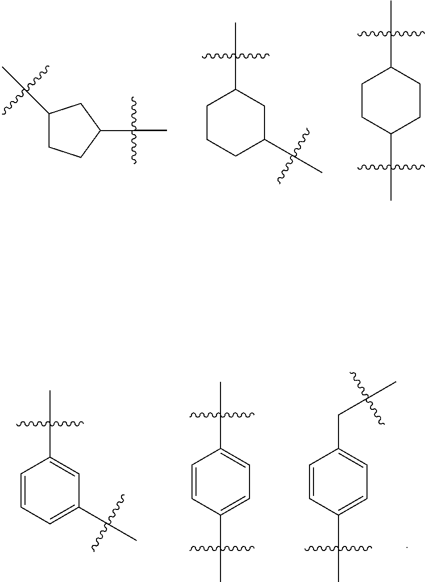 [SMILES: *C1CCC(*)C1 ; *C1CCCC(*)C1 ; *C1CCC(*)CC1 ; *c1cccc(*)c1 ; *c1ccc(*)cc1 ; *Cc1ccc(*)cc1]

In an embodiment, R₁ and R₂ are independently selected from H or CH₃. In another embodiment, R₁ and R₂ are both H. In another embodiment, R₁ and R₂ are both CH₃. In another embodiment, R₁ is H and R₂ is CH₃. In another embodiment, R₁ is selected from H or CH₃ and R₂ is selected from

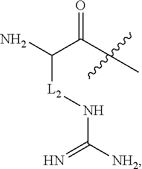

wherein L₂ is selected from —(CH₂)ₙ—, C₂-C₅ alkenylene, C₂-C₅ alkynylene, C₃-C₆ cycloalkylene, phenylene or benzylene, wherein n is selected from 1 to 5. In another embodiment, L₂ is —(CH₂)—. In another embodiment, L₂ is —(CH₂CH₂)—. In another embodiment, L₂ is —(CH₂CH₂CH₂)—. In another embodiment, L₂ is —(CH₂CH₂CH₂CH₂)—. In another embodiment, L₂ is —(CH₂CH₂CH₂CH₂CH₂)—. In another embodiment, L₂ is —(CH═CH)—. In another embodiment, L₂ is —(CH═CHCH₂)—. In another embodiment, L₂ is —(CH₂CH═CH)—. In another embodiment, L₂ is —(CH═CHCH₂CH₂)—. In another embodiment, L₂ is —(CH₂CH═CHCH₂)—. In another embodiment, L₂ is —(CH₂CH₂CH═CH)—. In another embodiment, L₂ is —(CH═CHCH₂CH₂CH₂)—. In another embodiment, L₂ is —(CH₂CH═CHCH₂CH₂)—. In another embodiment, L₂ is —(CH₂CH₂CH═CHCH₂)—. In another embodiment, L₂ is —(CH₂CH₂CH₂CH═CH)—. In another embodiment, L₂ is —(C≡C)—. In another embodiment, L₂ is —(C≡CCH₂)—. In another embodiment, L₂ is —(CH₂C≡C)—. In another embodiment, L₂ is —(C≡CCH₂CH₂)—. In another embodiment, L₂ is —(CH₂C≡CCH₂)—. In another embodiment, L₂ is —(CH₂CH₂C≡C)—. In another embodiment, L₂ is —(C≡CCH₂CH₂CH₂)—. In another embodiment, L₂ is —(CH₂CCCH₂CH₂)—. In another embodiment, L₂ is —(CH₂CH₂C≡CCH₂)—. In another embodiment, L₂ is —(CH₂CH₂CH₂C≡C)—. In another embodiment, R₁ is selected from H or CH₃ and R₂ is selected from

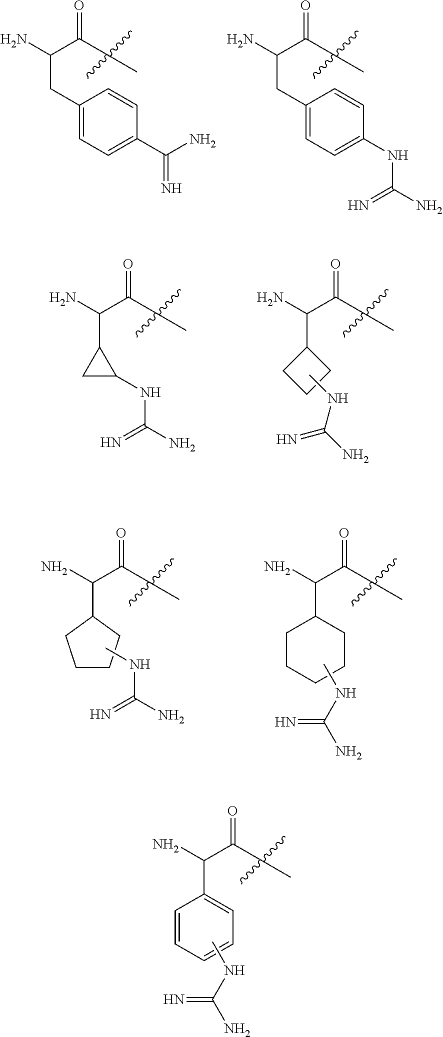

In another embodiment, R₁ is selected from H or CH₃ and R₂ is selected from

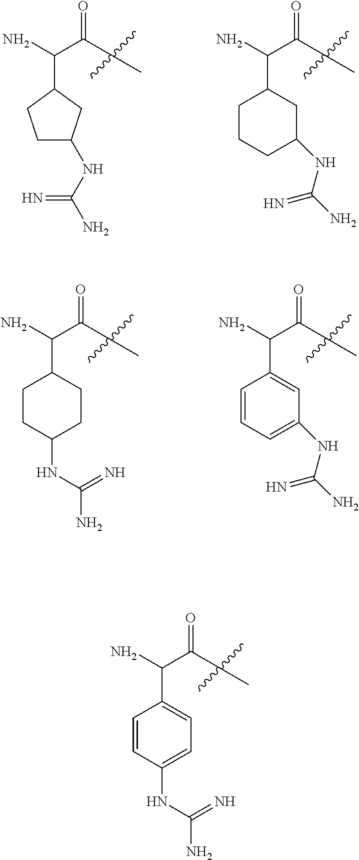 [SMILES: *C(=O)C(N)C1CCC(NC(=N)N)C1 ; *C(=O)C(N)C1CCCC(NC(=N)N)C1 ; *C(=O)C(N)C1CCC(NC(=N)N)CC1 ; *C(=O)C(N)c1cccc(NC(=N)N)c1 ; *C(=O)C(N)c1ccc(NC(=N)N)cc1]

In another embodiment, R₁ is selected from H or CH₃ and R₂ is selected from

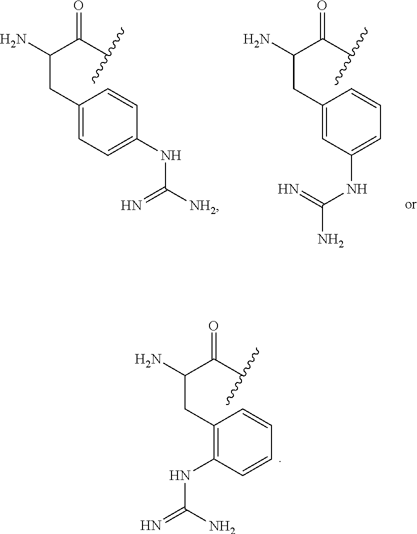 [SMILES: *C(=O)C(N)Cc1ccc(NC(=N)N)cc1 ; *C(=O)C(N)Cc1cccc(NC(=N)N)c1 ; "or" ; *C(=O)C(N)Cc1ccccc1NC(=N)N]

In an embodiment, R₃-R₈ are independently selected from H, Cl, I, Br or F; wherein at least one of R₃-R₈ is Cl, I, Br or F. In another embodiment, R₃ is selected from Cl, I, Br, or F. In another embodiment, R₄ is selected from Cl, I, Br, or F. In another embodiment, R₅ is selected from Cl, I, Br, or F. In another embodiment, R₆ is selected from Cl, I, Br, or F. In another embodiment, R₇ is selected from Cl, I, Br, or F. In another embodiment, R₈ is selected from Cl, I, Br, or F.

In one embodiment, R₃ and R₄ are independently selected from Cl, I, Br, or F. In another embodiment, R₃ and R₅ are independently selected from Cl, I, Br, or F. In another embodiment, R₆ and R₇ are independently selected from Cl, I, Br, or F. In another embodiment, R₆ and R₈ are independently selected from Cl, I, Br, or F. In another embodiment, R₃ and R₆ are independently selected from Cl, I, Br, or F. In another embodiment, R₃ and R₇ are independently selected from Cl, I, Br, or F. In another embodiment, R₄ and R₆ are independently selected from Cl, I, Br, or F. In another embodiment, R₄ and R₇ are independently selected from Cl, I, Br, or F.

In an embodiment, R₃ is Cl or Br. In another embodiment, R₄ is Cl or Br. In another embodiment, R₅ is Cl or Br. In another embodiment, R₆ is Cl or Br. In another embodiment, R₇ is Cl or Br.

In one embodiment, R₃ and R₄ are independently selected from Cl or Br. In another embodiment, R₃ and R₅ are independently selected from Cl or Br. In another embodiment, R₆ and R₇ are independently selected from Cl or Br. In another embodiment, R₆ and R₈ are independently selected from Cl or Br. In another embodiment, R₃ and R₆ are independently selected from Cl or Br. In another embodiment, R₄ and R₇ are independently selected from Cl or Br. In another embodiment, R₃ and R₇ are independently selected from Cl or Br. In another embodiment, R₄ and R₆ are independently selected from Cl or Br.

In one embodiment, R₃ and R₄ are each a Cl. In another embodiment, R₃ and R₅ are each a Cl. In another embodiment, R₆ and R₇ are each a Cl. In another embodiment, R₆ and R₈ are each a Cl. In another embodiment, R₃ and R₆ each a Cl. In another embodiment, R₄ and R₇ are each a Cl. In another embodiment, R₃ and R₇ are each a Cl. In another embodiment, R₄ and R₆ are each a Cl.

In an embodiment, R₉ is selected from

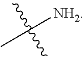 [SMILES: *N]

In another embodiment, R₉ is selected from

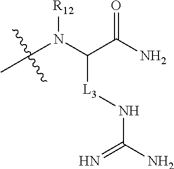

wherein L₃ is selected from —(CH₂)ₒ—, C₂-C₅ alkenylene, C₂-C₅ alkynylene, C₃-C₆ cycloalkylene, phenylene or benzylene, wherein o is selected from 1 to 5, wherein R₁₂ is selected from H or CH₃. In another embodiment, L₃ is —(CH₂)—. In another embodiment, L₃ is —(CH₂CH₂)—. In another embodiment, L₃ is —(CH₂CH₂CH₂)—. In another embodiment, L₃ is —(CH₂CH₂CH₂CH₂)—. In another embodiment, L₃ is —(CH₂CH₂CH₂CH₂CH₂)—. In another embodiment, L₃ is —(CH═CH)—. In another embodiment, L₃ is —(CH═CHCH₂)—. In another embodiment, L₃ is —(CH₂CH═CH)—. In another embodiment, L₃ is —(CH═CHCH₂CH₂)—. In another embodiment, L₃ is —(CH₂CH═CHCH₂)—. In another embodiment, L₃ is —(CH₂CH₂CH═CH)—. In another embodiment, L₃ is —(CH═CHCH₂CH₂CH₂)—. In another embodiment, L₃ is —(CH₂CH═CHCH₂CH₂)—. In another embodiment, L₃ is —(CH₂CH₂CH═CHCH₂)—. In another embodiment, L₃ is —(CH₂CH₂CH₂CH═CH)—. In another embodiment, L₃ is —(C≡C)—. In another embodiment, L₃ is —(C≡CCH₂)—. In another embodiment, L₃ is —(CH₂C≡C)—. In another embodiment, L₃ is —(C≡CCH₂CH₂)—. In another embodiment, L₃ is —(CH₂C≡CCH₂)—. In another embodiment, L₃ is —(CH₂CH₂C≡C)—. In another embodiment, L₃ is —(C≡CCH₂CH₂CH₂)—. In another embodiment, L₃ is —(CH₂C≡CCH₂CH₂)—. In another embodiment, L₃ is —(CH₂CH₂C≡CCH₂)—. In another embodiment, L₃ is —(CH₂CH₂CH₂C≡C)—.

In another embodiment, R₉ is selected from

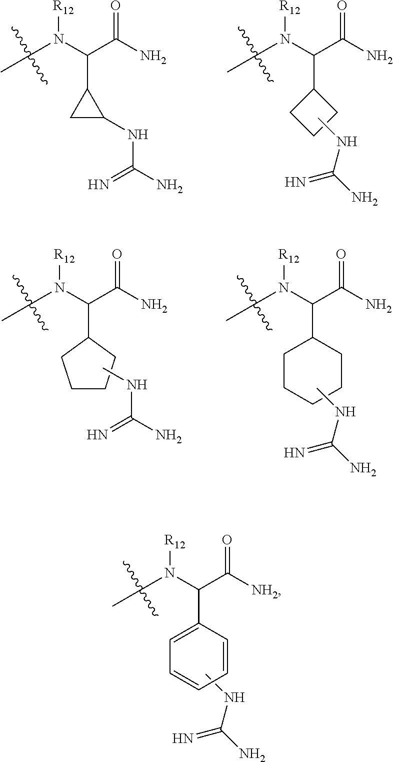

wherein R₁₂ is selected from H or CH₃.

In another embodiment, R₉ is selected from

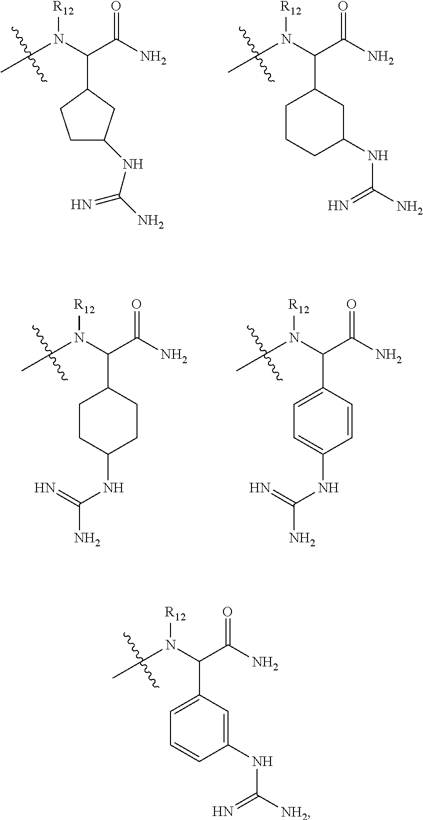

wherein R₁₂ is selected from H or CH₃.

In another embodiment, R₉ is selected from

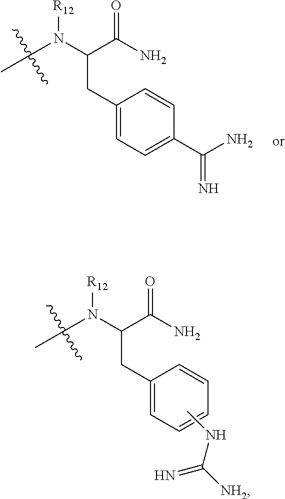

wherein R₁₂ is selected from H or CH₃.

In another embodiment, R₉ is selected from

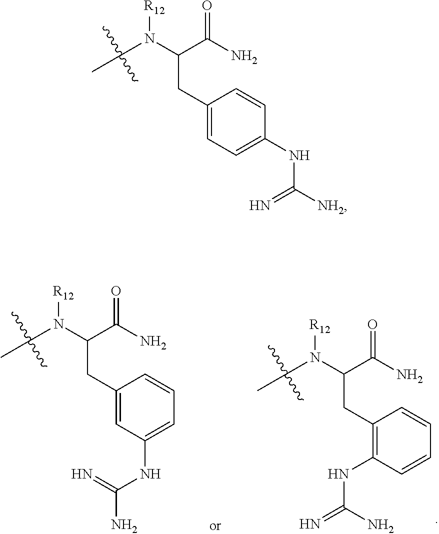

wherein R₁₂ is selected from H or CH₃.

In another embodiment, R₉ is selected from

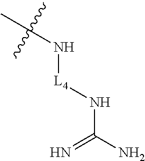

wherein L₄ is selected from —(CH₂)ₚ—, C₂-C₅ alkenylene, C₂-C₅ alkynylene, C₃-C₆ cycloalkylene, phenylene or benzylene, wherein p is selected from 1 to 5. In another embodiment, L₄ is —(CH₂)—. In another embodiment, L₄ is —(CH₂CH₂)—. In another embodiment, L₄ is —(CH₂CH₂CH₂)—. In another embodiment, L₄ is —(CH₂CH₂CH₂CH₂)—. In another embodiment, L₄ is —(CH₂CH₂CH₂CH₂CH₂)—. In another embodiment, L₄ is —(CH═CH)—. In another embodiment, L₄ is —(CH═CHCH₂)—. In another embodiment, L₄ is —(CH₂CH═CH)—. In another embodiment, L₄ is —(CH═CHCH₂CH₂)—. In another embodiment, L₄ is —(CH₂CH═CHCH₂)—. In another embodiment, L₄ is —(CH₂CH₂CH═CH)—. In another embodiment, L₄ is —(CH═CHCH₂CH₂CH₂)—. In another embodiment, L₄ is —(CH₂CH═CHCH₂CH₂)—. In another embodiment, L₄ is —(CH₂CH₂CH═CHCH₂)—. In another embodiment, L₄ is —(CH₂CH₂CH₂CH═CH)—. In another embodiment, L₄ is —(C≡C)—. In another embodiment, L₄ is —(C≡CCH₂)—. In another embodiment, L₄ is —(CH₂C≡C)—. In another embodiment, L₄ is —(C≡CCH₂CH₂)—. In another embodiment, L₄ is —(CH₂C≡CCH₂)—. In another embodiment, L₄ is —(CH₂CH₂C≡C)—. In another embodiment, L₄ is —(C≡CCH₂CH₂CH₂)—. In another embodiment, L₄ is —(CH₂C≡CCH₂CH₂)—. In another embodiment, L₄ is —(CH₂CH₂C≡CCH₂)—. In another embodiment, L₄ is —(CH₂CH₂CH₂C≡C)—. In another embodiment, R₉ is selected from

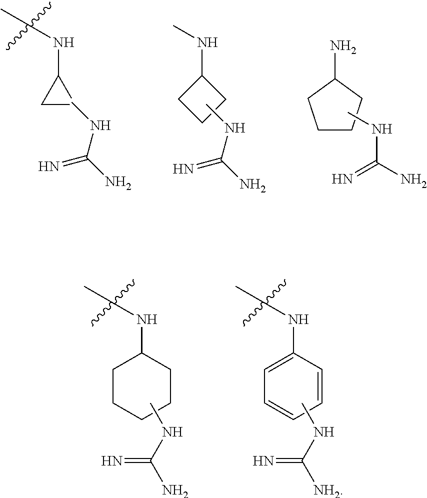

In another embodiment, R₉ is selected from

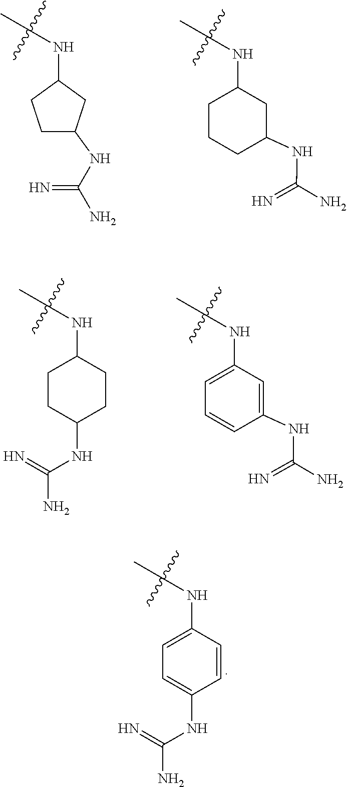 [SMILES: *NC1CCC(NC(=N)N)C1 ; *NC1CCCC(NC(=N)N)C1 ; *NC1CCC(NC(=N)N)CC1 ; *Nc1cccc(NC(=N)N)c1 ; *Nc1ccc(NC(=N)N)cc1]

In an embodiment, R₁₀ and R₁₁ are H. In another embodiment, R₁₀ is CH₃ and R₁₁ is H. In another embodiment, R₁₀ is H and R₁₁ is CH₃. In an embodiment, R₁₀ and R₁₁ are CH₃.

In an embodiment, R₁ and R₂ are H; L₁ is —(CH₂)ₘ—, wherein m is selected from 1 to 5; wherein R₃-R₈ are independently H, Cl, I, Br or F; wherein at least one of R₃-R₈ is Cl, I, Br or F; R₉ is

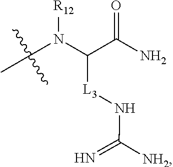

wherein L₃ is —(CH₂)ₒ—, wherein o is selected from 1 to 5; and R₁₀-R₁₂ are H.

In an embodiment, R₁ is H, R₂ is

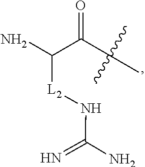

wherein L₂ is —(CH₂)ₙ—, and wherein n is selected from 1 to 5; L₁ is —(CH₂)ₘ—, wherein m is selected from 1 to 5; wherein R₃-R₈ are independently H, Cl, I, Br or F; wherein at least one of R₃-R₈ is Cl, I, Br or F; R₉ is

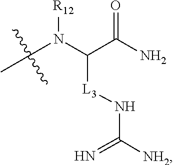

wherein L₃ is —(CH₂)ₒ—, wherein o is selected from 1 to 5; and R₁₀-R₁₂ are H.

In an embodiment, R₁ and R₂ are H; L₁ is —(CH₂)ₘ—, wherein m is selected from 1 to 5; R₄ and R₇ are Cl or Br; R₃, R₅, R₆, and R₈ are H; R₉ is

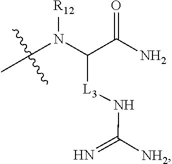

wherein L₃ is —(CH₂)ₒ—, wherein o is selected from 1 to 5; and R₁₀-R₁₂ are H.

In an embodiment, R₁ is H, R₂ is

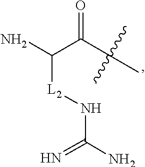

wherein L₂ is —(CH₂)ₙ—, and wherein n is selected from 1 to 5; L₁ is —(CH₂)ₘ—, wherein m is selected from 1 to 5; R₄ and R₇ are Cl or Br; R₃, R₅, R₆, and R₈ are H; R₉ is

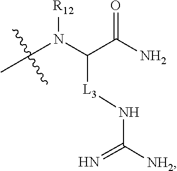

wherein L₃ is —(CH₂)—, wherein o is selected from 1 to 5; and R₁₀-R₁₂ are H.

In an embodiment, there is provided a compound of formula (I):

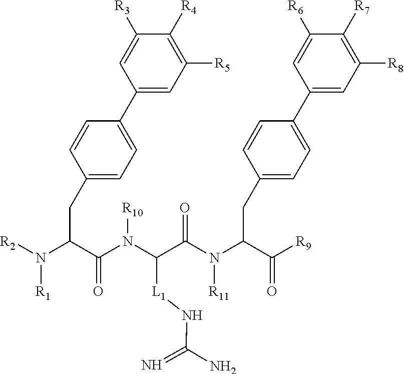

or a pharmaceutically acceptable salt or solvate thereof;
wherein L₁, L₂, L₃, L₄, R₁, R₂, R₉, R₁₀, R₁₁ and R₁₂ are as disclosed herein;
wherein R₃, R₅, R₆ and R₈ are H; and
wherein R₄ and R₇ are independently Cl, I, Br or F.

In an embodiment, there is provided a compound of formula (I):

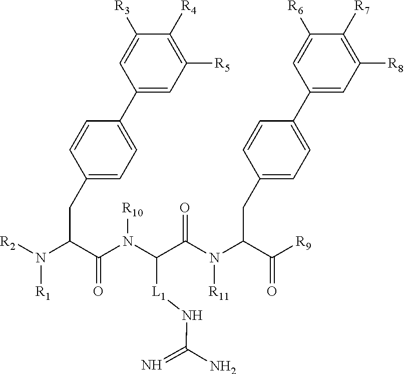

or a pharmaceutically acceptable salt or solvate thereof;
wherein L₁, L₂, L₃, L₄, R₁, R₂, R₉, R₁₀, R₁₁ and R₁₂ are as disclosed herein;
wherein R₃, R₅, R₆ and R₈ are H; and
wherein R₄ and R₇ are independently Cl.

In one aspect, there is provided a process for making compounds of formula (I):

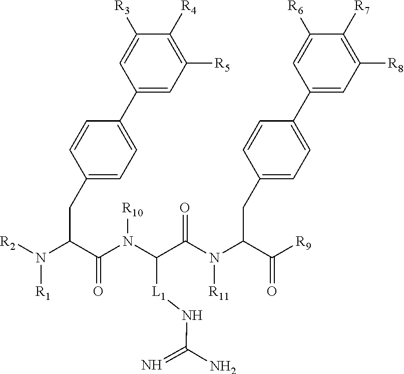

or a pharmaceutically acceptable salt or solvate thereof;
wherein L₁ is —(CH₂)ₘ—, C₂-C₅ alkenylene, C₂-C₅ alkynylene, C₃-C₆ cycloalkylene, phenylene or benzylene, wherein m is selected from 1 to 5;
wherein R₁ and R₂ are independently selected from H or CH₃, or
R₁ is selected from H or CH₃ and R₂ is

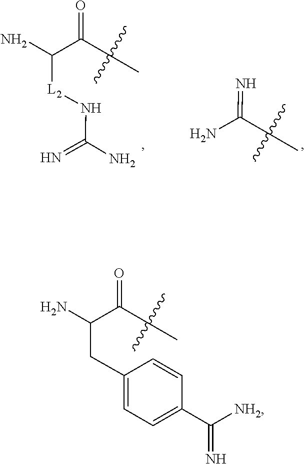

wherein L₂ is —(CH₂)ₙ—, C₂-C₅ alkenylene, C₂-C₅ alkynylene, C₃-C₆ cycloalkylene, phenylene or benzylene, wherein n is selected from 1 to 5;
wherein R₃-R₈ are independently H, Cl, I, Br or F;
wherein at least one of R₃-R₈ is Cl, I, Br or F;
wherein R₉ is

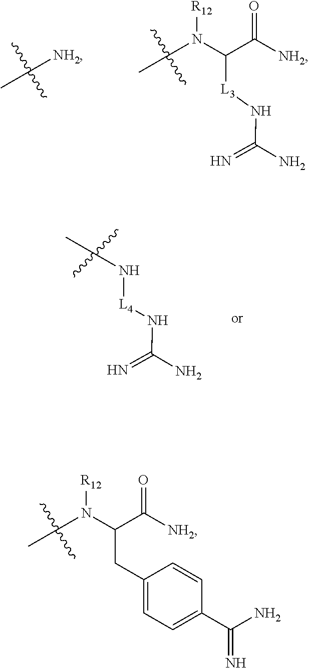

wherein L₃ is —(CH₂)ₒ—, C₂-C₅ alkenylene, C₂-C₅ alkynylene, C₃-C₆ cycloalkylene, phenylene or benzylene, wherein o is selected from 1 to 5;
wherein L₄ is —(CH₂)ₚ—, C₂-C₅ alkenylene, C₂-C₅ alkynylene, C₃-C₆ cycloalkylene, phenylene or benzylene, wherein p is selected from 1 to 5;
wherein R₁₀, R₁₁ and R₁₂ are independently H or —CH₃, by reacting following compounds of formula (II):

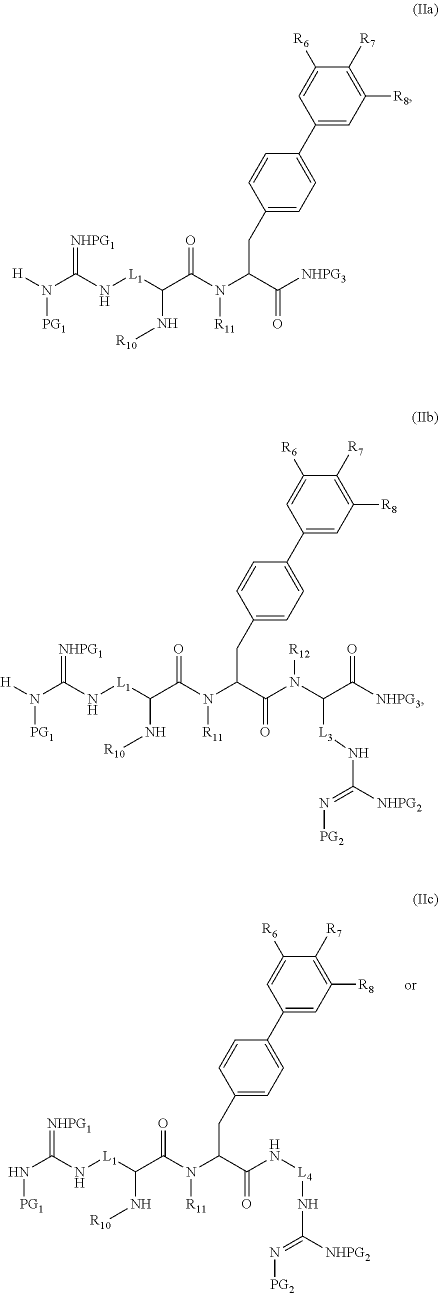

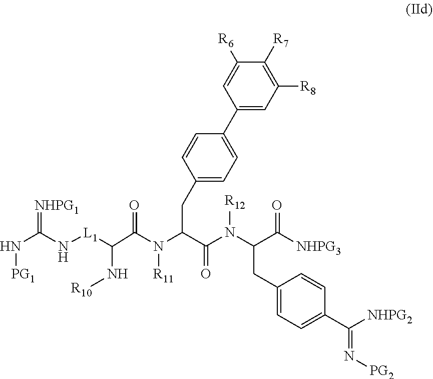

wherein R₆, R₇, R₈, R₁₀, R₁₁, R₁₂, L₁, L₃ and L₄ have the meaning given above; wherein PG₁, PG₂ and PG₃ can be any protecting group such as t-Boc or Pbf; in the presence of an amide/peptide coupling reagent with compounds of the formula (III)

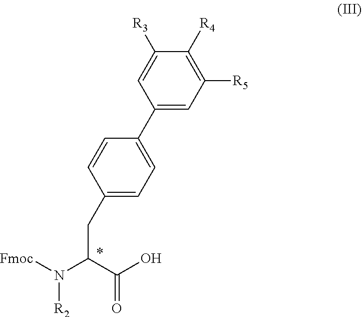

wherein R₂-R₅ are defined as mentioned above,
wherein * denotes an stereogenic carbon;
and deprotection with an acid.

The person skilled in the art would understand that one Pbf is sufficient to protect the guanidine group while two t-Bocs are required to protect the guanidine group. In certain embodiments, the guanidine group is protected when both PG₁ are t-Bocs. In certain embodiments, the guanidine group is protected when both PG₂ are t-Bocs. In certain embodiments, the guanidine group is protected when both PG₁ is replaced by a single Pbf. In certain embodiments, the guanidine group is protected when both PG₂ is replaced by a single Pbf.

Compounds of formula (II) can be made by the process wherein

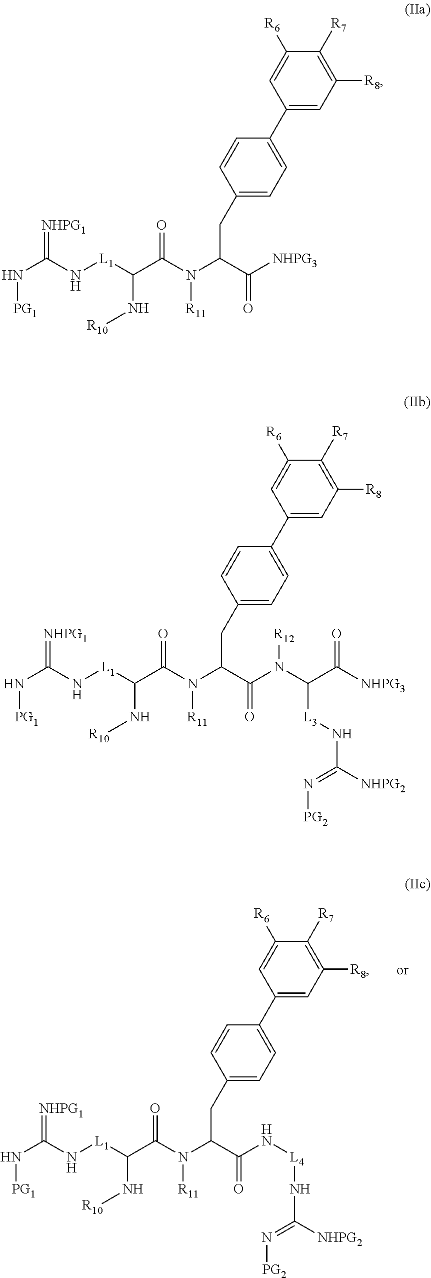

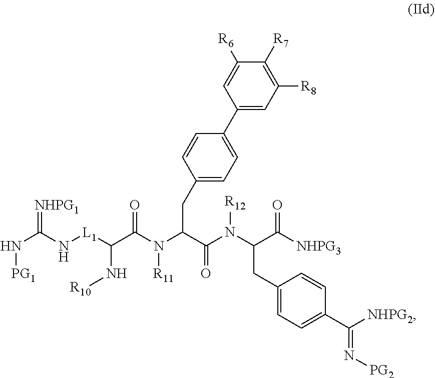

wherein R₆, R₇, R₈, R₁₀, R₁₁, R₁₂, L₁, L₃ and L₄ have the meaning given above; wherein PG₁, PG₂ and PG₃ can be any protecting group such as t-Boc or Pbf; by reacting following compounds of formula (IV):

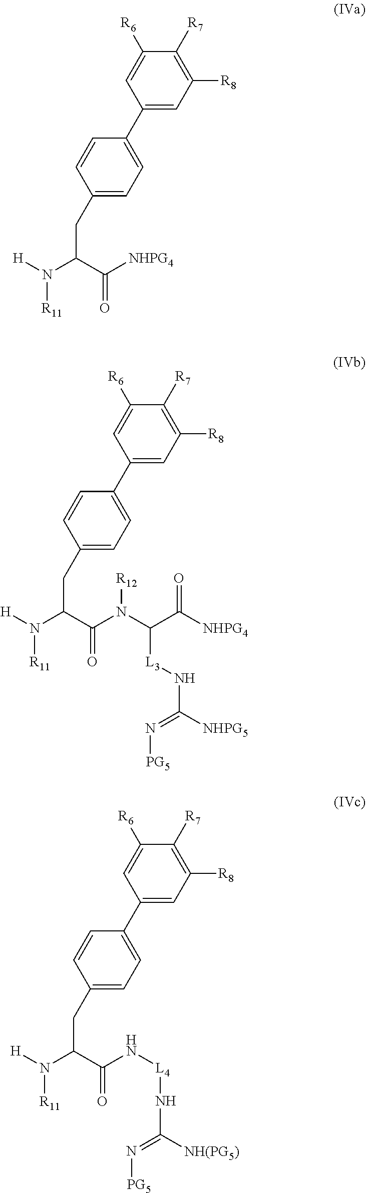

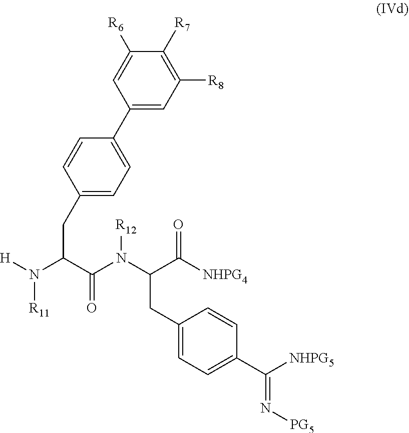

wherein R₆, R₇, R₈, R₁₁, R₁₂, L₃ and L₄ have the meaning given above; wherein PG₄ and PG₅ can be any protecting group such as t-Boc or Pbf;
in the presence of an amide/peptide coupling reagent with compounds of the formula (V)

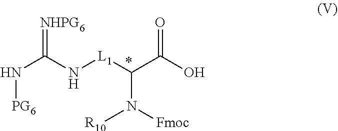

wherein R₁₀ and L₁ are defined as mentioned above; wherein PG₆ can be any protecting group such as t-Boc or Pbf;
wherein * denotes an stereogenic carbon;
and deprotection with an acid.
In certain embodiments, the guanidine group is protected when both PG₅ are t-Boc. In certain embodiments, the guanidine group is protected when both PG₆ are t-Boc. In certain embodiments, the guanidine group is protected when both PG₅ is replaced by a single Pbf. In certain embodiments, the guanidine group is protected when both PG₆ is replaced by a single Pbf.

Compounds of formula (IV) can be made by the process wherein

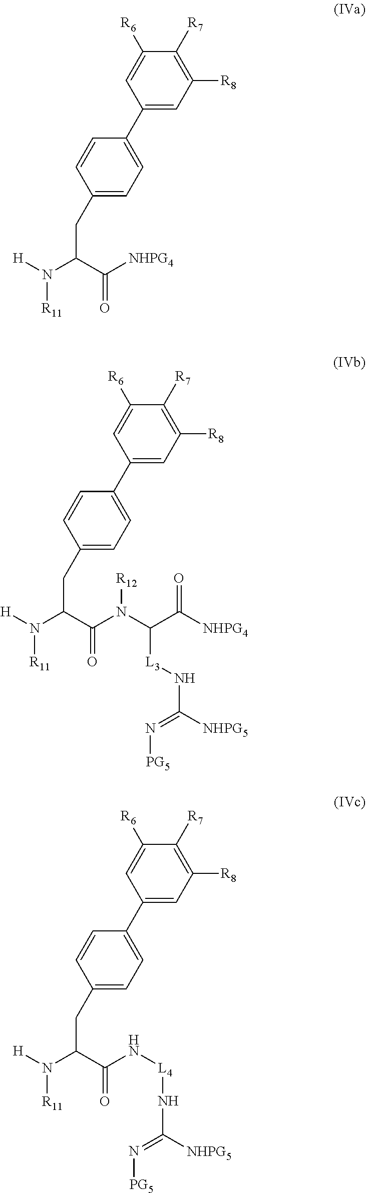

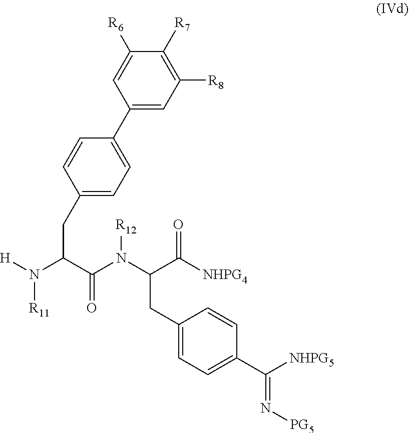

wherein R₆, R₇, R₈, R₁₁, R₁₂, L₃ and L₄ have the meaning given above; wherein PG₄ and PG₅ can be any protecting group such as t-Boc or Pbf;
by reacting following compounds of formula (VI):

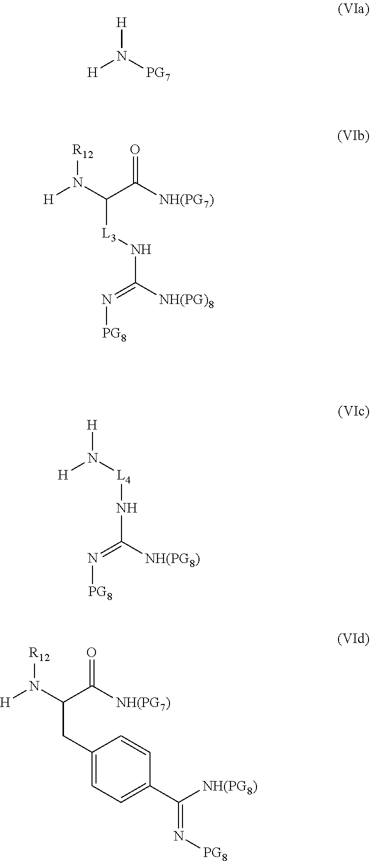

wherein R₁₂, L₃ and L₄ have the meaning given above; wherein PG₇ and PG₈ can be any protecting group such as t-Boc or Pbf;
in the presence of an amide/peptide coupling reagent with compounds of the formula (VII)

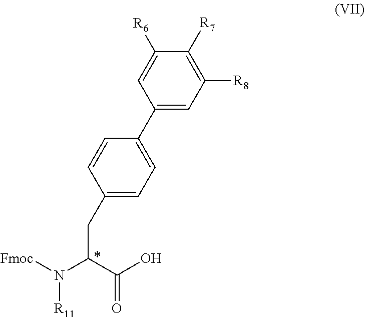

wherein R₆, R₇, R₈ and R₁₁ have the meaning given above;
wherein * denotes an stereogenic carbon; and deprotection with an acid.
In certain embodiments, the guanidine group is protected when both PG₈ are t-Boc. In certain embodiments, the guanidine group is protected when both PG₈ is replaced by a single Pbf.

Compounds of formula (VIb) and (VIc) can be made by reacting following compounds of formula (VIII):

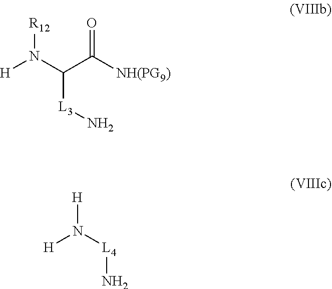

wherein R₁₂, L₃ and L₄ have the meaning given above; wherein PG₉ can be any protecting group such as t-Boc or Pbf;
with N,N′-di-Boc-S-methylisothiourea.

Compounds of formula (VIIb) can be obtained as known from the chemical literature.

The person skilled in the art will understand that compounds of formula (I) shown below:

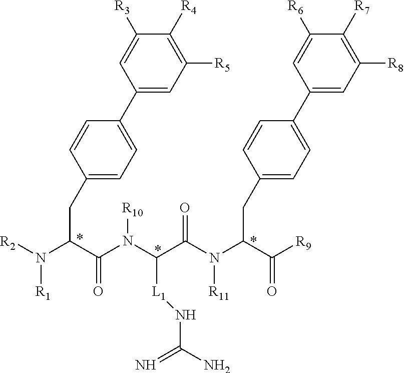

has at least 3 stereogenic carbon centers as denoted by *. The person skilled in the art will understand that more than 3 stereogenic carbon centers may exist, depending on what R₂ and R₉ are.

The present invention encompasses all stereoisomers available of compounds of formula (I). In an embodiment, compounds of formula (I) can be produced by L-enantiomers. In another embodiment, compounds of formula (I) can be produced by L and D-enantiomers. In another embodiment, compounds of formula (I) can be produced by D-enantiomers. Without wishing to be bound to theory, it is believed that peptides made from a combination of L and D-enantiomers can lead to an increase in peptide metabolic stability due to higher resistance against proteolytic degradation by human proteases found in skin, blood, body fluids and tissues, as well as bacterial proteases. When only D-enantiomers are used, it has been observed that this leads to a further increase in peptide's plasma stability.

In certain embodiments, it has been observed that when D-enantiomers are used, there is an improved bioactivity relative to the L-enantiomers.

A skilled person would also understand that the natural or unnatural amino acids of the compounds of the present invention may be substituted with natural or unnatural β-homo amino acids while retaining their bioactivity.

In one embodiment, the compounds of the present invention have improved plasma stability. The compounds may also have improved metabolic stability. The compounds may also have improved pharmacokinetic properties.

The process for making the compounds of formula (I) is described now in more detail.

In a first reaction step known and commercially available diamines of the formula (VIII) can be reacted with N,N′-di-Boc-S-methylisothiourea in the presence of a base. This reagent is commercially available from Sigma-Aldrich (Merck). Bases can be customary acid acceptors such as tertiary amines, preferably N,N-disopropylethylamine. Suitable solvents include inert organic solvents such as hydrocarbons, preferably methylene dichloride (dichloromethane).

The reaction temperatures in this process step can be varied in a relatively wide range. In general the process is carried out at temperatures of 0 to 100° C., preferably 15 to 60° C., most preferably at room temperature.

When carrying out this process step the starting materials of formula (VIII) and the reagent are generally each employed in approximately equal amount. It may be beneficial to use the diamine of formula (VIII) in excess to the reagent.

Work up can be done by customary separation methods, preferably flash chromatography and evaporation of the solvents.

In a second reaction step the obtained compounds of the formula (VI) can be reacted with a compound of the formula (VII). Compounds of the formula (VII) are known or can be prepared according to known methods. For instance one of such compounds is commercially available from Merck Millipore and GL Biochem China, Anaspec, Bachem, Chempep, Iris Biotech, Polypeptides or Sigma-Aldrich (Merck) as “Fmoc-4-phenyl-Phe-OH”.

The amide/peptide coupling reagent can be customary coupling reagents such as 2-(1H-Benzotriazole-1-yl)-1,1,3,3-tetramethyluronium hexafluorophosphate (HBTU). Other suitable coupling reagents include N,N′-Dicyclohexylcarbodiimide (DCC), (N,N′-Diisopropylcarbodiimide (DIC), (1-[Bis(dimethylamino)methylene]-1H-1,2,3-triazolo[4,5-bjpyridinium 3-oxid hexafluorophosphate (HATU), 2-(6-Chloro-1H-benzotriazole-1-yl)-1,1,3, 3-tetramethylaminium hexafluorophosphate (HCTU), benzotriazol-1-yl-oxytripyrrolidinophosphonium hexafluorophosphate (PyBop), 6-Chloro-benzotriazole-1-yloxy-tris-pyrrolidinophosphonium hexafluorophosphate (Pyclock), Ethyl 2-Cyano-2-(hydroxyimino) acetate (Oxyma), N-(Benzenesulfonyl)-L-prolyl-L-O-(1-pyrrolidinylcarbonyl)tyrosine sodium salt (BOP), N,N′-bis(2-oxo-3-oxazolidinyl)-phosphinic chloride (BOP-Cl), 1,1′-carbonyldiimidazole (CDI), 1-Cyano-2-ethoxy-2-oxoethylidenaminooxy)dimethylamino-morpholino-carbenium hexafluorophosphate (COMU), 3-(Diethoxyphosphoryloxy)-1,2,3-benzotriazin-4(3H)-one (DEPBT), 1-[3-(dimethylamino)propyl]-3-ethylcarbodiimide hydrochloride (EDC), [(7-azabenzotriazol-1-yl)oxy]tris-(pyrrolidino)phosphonium hexafluorophosphate (PyAOP), [Ethyl cyano(hydroxyimino)acetato-O2]tri-1-pyrrolidinylphosphonium hexafluorophosphate (PyOxim), O-(7-azabenzotriazol-1-yl)-1,1,3,3-tetramethyluroniumtetrafluoroborate (TATU), O-benzotriazol-1-yl-1,1,3,3-tetramethyluronium tetrafluoroborate (TBTU), O-[cyano(ethoxycarbonyl)methyleneamino]-N,N,N,N′-tetramethyluronium tetrafluoroborate (TOTU), or N,N,N′,N′-Tetramethyl-O—(N-succinimidyl)uronium tetrafluoroborate (TSTU). Preferably these coupling reagents are used in the presence of a base such as for instance a tertiary amine, preferably N, N-Diisopropylamine. Anti-racemization additives can also be added such as 1-hydroxy-7-azabenzotriazole (HOAt) and 1-hydroxybenzotriazole (HOBt).

The reaction temperatures in this process step can be varied in a relatively wide range. In general the process is carried out at temperatures of 0 to 100° C., preferably 15 to 60° C., most preferably at room temperature.

When carrying out this process step the starting materials of formula (VI) and the compound of formula (VII) are generally each employed in approximately equal amount. It may be beneficial to use the compound of formula (VII) in small excess.

Work up can be done by customary separation methods, preferably by washing steps and an evaporation of the solvent. Dissolution and further post-reaction with a base, such as 1,8-Diazabicyclo [5.4.0] undec-7-ene (DBU), at room temperature and flash chromatography is possible.

In a third reaction step the obtained compounds of the formula (IV) can be reacted with a compound of the formula (V). Compounds of the formula (V) are known or can be prepared according to known methods. For instance one of such compounds is commercially available from Merck Millipore or GL Biochem, Anaspec, Bachem, Chempep, Iris Biotech, Polypeptides or Sigma-Aldrich (Merck) as “Fmoc-Arg(Pbf)-OH” or Fmoc-Arg(Boc)-2-OH.

The amide/peptide coupling reagent can be customary coupling reagents such as 2-(1H-Benzotriazole-1-yl)-1,1,3,3-tetramethyluronium hexafluorophosphate (HBTU). Other suitable coupling reagents include N,N′-Dicyclohexylcarbodiimide (DCC), (N,N′-Diisopropylcarbodiimide (DIC), (1-[Bis(dimethylamino)methylene]-1H-1,2,3-triazolo [4,5-b]pyridinium 3-oxid hexafluorophosphate (HATU), 2-(6-Chloro-1H-benzotriazole-1-yl)-1,1,3,3-tetramethylaminium hexafluorophosphate (HCTU), benzotriazol-1-yl-oxytripyrrolidinophosphonium hexafluorophosphate (PyBop), 6-Chloro-benzotriazole-1-yloxy-tris-pyrrolidinophosphonium hexafluorophosphate (Pyclock) or Ethyl 2-Cyano-2-(hydroxyimino) acetate (Oxyma). Preferably these coupling reagents are used in the presence of a base such as for instance a tertiary amine, preferably N,N-Diisopropylamine.

Suitable solvents include inert organic solvents such as dimethylformamide.

The reaction temperatures in this process step can be varied in a relatively wide range. In general the process is carried out at temperatures of 0 to 100° C., preferably 15 to 60° C., most preferably at room temperature.

When carrying out this process step the starting materials of formula (IV) and the compound of formula (V) are generally each employed in approximately equal amount. It may be beneficial to use the compound of formula (V) in excess.

Work up can be done by customary separation methods, preferably by washing steps and an evaporation of the solvent. Dissolution and further post reaction with a base, such as 1,8-Diazabicyclo [5.4.0] undec-7-ene (DBU) or piperidine, at room temperature and flash chromatography is possible.

In a fourth reaction step the obtained compounds of the formula (II) can be reacted with a compound of the formula (III). Compounds of the formula (III) are known or can be prepared according to known methods. For instance one of such compounds is commercially available from Merck Millipore or GL Biochem, Anaspec, Bachem, Chempep, Iris Biotech, Polypeptides or Sigma-Aldrich (Merck) as “Fmoc-4-phenyl-Phe-OH” or “Fmoc-Bip-OH”. It can also be bought from Creosalus Advanced ChemTech.

The amide/peptide coupling reagent can be customary coupling reagents such as 2-(1H-Benzotriazole-1-yl)-1,1,3,3-tetramethyluronium hexafluorophosphate (HBTU). Other suitable coupling reagents include N,N′-Dicyclohexylcarbodiimide (DCC), [N,N′-Diisopropylcarbodiimide (DIC), (1-[Bis(dimethylamino)methylene]-1H-1,2,3-triazolo [4,5-b]pyridinium 3-oxid hexafluorophosphate (HATU), 2-(6-Chloro-1H-benzotriazole-1-yl)-1,1,3,3-tetramethylaminium hexafluorophosphate (HCTU), benzotriazol-1-yl-oxytripyrrolidinophosphonium hexafluorophosphate (PyBop), 6-Chloro-benzotriazole-1-yloxy-tris-pyrrolidinophosphonium hexafluorophosphate (Pyclock), Ethyl 2-Cyano-2-(hydroxyimino) acetate (Oxyma), N-(Benzenesulfonyl)-L-prolyl-L-O-(1-pyrrolidinylcarbonyl)tyrosine sodium salt (BOP), N,N′-bis(2-oxo-3-oxazolidinyl)-phosphinic chloride (BOP-Cl), 1,1′-carbonyldiimidazole (CDI), 1-Cyano-2-ethoxy-2-oxoethylidenaminooxy)dimethylamino-morpholino-carbenium hexafluorophosphate (COMU), 3-(Diethoxyphosphoryloxy)-1,2,3-benzotriazin-4(3H)-one (DEPBT), 1-[3-(dimethylamino)propyl]-3-ethylcarbodiimide hydrochloride (EDC), [(7-azabenzotriazol-1-yl)oxy]tris-(pyrrolidino)phosphonium hexafluorophosphate (PyAOP), [Ethyl cyano(hydroxyimino)acetato-O2]tri-1-pyrrolidinylphosphonium hexafluorophosphate (PyOxim), O-(7-azabenzotriazol-1-yl)-1,1,3,3-tetramethyluroniumtetrafluoroborate (TATU), O-benzotriazol-1-yl-1,1,3,3-tetramethyluronium tetrafluoroborate (TBTU), O-[cyano(ethoxycarbonyl)methyleneamino]-N,N,N,N′-tetramethyluronium tetrafluoroborate (TOTU), or N,N,N′,N′-Tetramethyl-O—(N-succinimidyl)uronium tetrafluoroborate (TSTU). Preferably these coupling reagents are used in the presence of a base such as for instance a tertiary amine, preferably N,N-Diisopropylamine. Anti-racemization additives can also be added such as 1-hydroxy-7-azabenzotriazole (HOAt) and 1-hydroxybenzotriazole (HOBt).

Suitable solvents include inert organic solvents such as dimethylformamide.

The reaction temperatures in this process step can be varied in a relatively wide range. In general the process is carried out at temperatures of 0 to 100° C., preferably 15 to 60° C., most preferably at room temperature.

When carrying out this process step the starting materials of formula (IV) and the compound of formula (V) are generally each employed in approximately equal amount. It may be beneficial to use the compound of formula (V) in excess.

Work up can be done by customary separation methods, preferably by washing steps and an evaporation of the solvent. Dissolution and further post-reaction with a base, such as diazabicycloundecene (DBU) or piperidine, at room temperature and flash chromatography is possible.

The compounds of the formula (I) can be obtained from their precursors by reaction with a strong organic acid such as trifluoroacetic acid. Such organic acids must be able to remove the Pbf and Boc moieties.

The reaction temperatures in this process step can be varied in a relatively wide range. In general the process is carried out at temperatures of 0 to 100° C., preferably 15 to 60° C., most preferably at room temperature.

Work up is done by customary separation methods, preferably by evaporation of the solvent, re-dissolution, chromatography and HPLC.

Some of the comparators can be prepared according to WO2015112093, the entire content of which is incorporated herein by reference.

Other compounds disclosed herein also can be synthesized analogous to the compounds of Formula (I) or can be synthesized utilizing known methodologies disclosed in texts well known to those skilled in the art such as Amino acids, Peptides and Proteins in Organic Chemistry, Ed. A. B. Hughes, vol. 4; Wiley-VCH, Germany, 2011, or Coin I, Beyermann M, Bienert M. Solid-phase peptide synthesis: from standard procedures to the synthesis of difficult sequences. Nat Protoc. 2007; 2(12):3247-56.

The compounds disclosed herein may be made by solid-phase peptide synthesis methods as described above. A skilled person would also be able to make the compounds using solution-phase synthesis methods that are known in the art.

In one aspect, there is provided a pharmaceutical composition comprising a compound of formula (I) or a pharmaceutically acceptable salt or solvate thereof, and a pharmaceutically acceptable excipient.

The compounds of the invention may exist in a continuum of solid states ranging from fully amorphous to fully crystalline. Compounds of the invention may also exist in both unsolvated and solvated forms. The term “solvate” is used herein to describe a molecular complex comprising the compound of the invention and a stoichiometric amount of one or more pharmaceutically acceptable solvent molecules. The term “hydrate” is employed when said solvent is water. A pharmaceutically acceptable acid addition salt may be readily prepared by using a desired acid as appropriate. Other non-pharmaceutically acceptable acid addition salts may be used, for example in the isolation of compounds of the invention, and are included within the scope of this invention. Typically an acid addition salt can be formed by reaction of a compound of formula (I) with a suitable inorganic or organic acid (such as acetic, benzoic, citric, hydrobromic, hydrochloric, formic, fumaric, maleic, methanesulfonic, naphthalenesulfonic, nitric, oxalic, p-toluenesulfonic, phosphoric, succinic, sulfuric, tartaric, or trifluoroacetic acid), optionally in a suitable solvent such as an organic solvent, to give the salt which is usually isolated for example by crystallisation and filtration. Thus, a pharmaceutically acceptable acid addition salt of a compound of formula (I) can be for example a acetate, benzoate, citrate, hydrobromide, hydrochloride, formate, fumarate, maleate, methanesulfonate, naphthalenesulfonate, nitrate, oxalate, p-toluenesulfonate phosphate, succinate, sulfate, tartrate, or trifluoroacetate salt.

The invention includes within its scope all possible stoichiometric and non-stoichiometric forms of the salts of the compounds of formula (I) and is not limited to those specifically mentioned.

Compounds of the present invention can form addition salts, reaction of the amino substituent of formula (I) with a suitable acid. Pharmaceutically acceptable salts of the compounds of formula (I) include the acid salts addition of them.

Without wanting to be bound by theory it is believed that the hydrophobic nature of the halogens at R₃-R₈ increases the affinity of the peptide for the bacteria's membrane and thus result in an improved membrane disrupting activity.

In some embodiments, the compounds of the invention show a particular surprising high activity against the bacteria selected from Staphylococcus aureus, Streptococcus pyogenes, Streptococcus pneumoniae, and Propionibacterium acnes. In some embodiments, the compounds of the invention show a particular surprising high activity against Gram-positive bacteria.

The compounds of the present invention can have potent bactericidal activities against strains that are resistant to penicillin-type antibiotics, such as Methicillin, and even Vancomycin. The compounds can also have potent bactericidal activities against strains that are resistant to Mupirocin, Linezolid, Retapamulin or Tigecycline. The compounds can be effective in combating these bacteria at surprisingly low micro molar levels such as 6.25 μM or less measured as MIC values (including low MIC values of about 1 μM).

In certain embodiments, the compounds are bactericidal against Gram-positive bacteria, including Staphylococcus aureus strains ATCC-BAA-1556, ATCC-BAA-1708, ATCC-BAA-1750 (USA200), ATCC-BAA-1681 (USA100) ATCC-BAA-1756 (USA300), ATCC-BAA-1707 (USA400), ATCC-BAA-2762 (EMRSA15, ST22), ATCC-BAA-1754 (ST45), ATCC-33592 (ST239), ATCC-700699 (VISA), ATTC-29213, RN 4220, ATCC-BAA-44, ATCC-BAA-1720, ATCC-BAA-2094, ATCC-33591 and ATCC-BAA-1680.

In one embodiment, the compounds are bactericidal against Staphylococcus aureus (SA) strains. In one embodiment, the compounds are bactericidal against Mupirocin-resistant MRSA strains and Mupirocin-susceptible MRSA strains. The Mupirocin-resistant MRSA strains include ATCC-BAA-1556, ATCC-BAA-1708 and ATCC-BAA-1750 (USA200). The Mupirocin-susceptible strains include ATCC-BAA-1681 (USA100) ATCC-BAA-1756 (USA300), ATCC-BAA-1707 (USA400), ATCC-BAA-2762 (EMRSA15, ST22), ATCC-BAA-1754 (ST45), ATCC-33592 (ST239) and ATCC-700699 (VISA).

In some embodiments, the compounds of the formula (I) or a pharmaceutically acceptable salt or solvate thereof are particularly useful for the treatment of diseases, disorders or conditions caused by bacteria.

In one embodiment, the compounds of formula (I) are extremely effective as antibacterial, advantageously showing potent bactericidal activities against MRSA.

In one embodiment, there is provided a method of treating a disease, disorder or condition, wherein the disease, disorder or condition is a bacterial infection, such as for instance a skin infection (e.g. boils, cuts, cellulitis, surgical wounds, impetigo), a blood infection, a respiratory disease (e.g. sinusitis, pneumonia), nasal decolonization, food poisoning or any other life-threatening systemic disease, in a subject in need of such treatment, comprising administering to said subject a compound of the formula (I) or a pharmaceutically acceptable salt or solvate thereof.

In one embodiment, there is provided a method of preventing a disease, disorder or condition, wherein the disease, disorder or condition is a bacterial infection, such as for instance a skin infection (e.g. boils, cuts, cellulitis, surgical wounds, impetigo), a blood infection, a respiratory disease (e.g. sinusitis, pneumonia), nasal decolonization, food poisoning or any other life-threatening systemic disease, in a subject in need of such treatment, comprising administering to said subject a compound of the formula (I) or a pharmaceutically acceptable salt or solvate thereof.

In one embodiment, there is provided a method of treating or preventing an acne condition, wherein the acne condition is a bacterial infection, in a subject in need of such treatment, comprising administering to said subject a compound of the formula (I) or a pharmaceutically acceptable salt or solvate thereof. In one embodiment, the bacterial infection is caused by Propionibacterium acnes.

In an embodiment, there is provided a method of disinfecting a surface against bacterial contamination by applying to said surface a compound of the formula (I) or an acceptable salt or solvate thereof. In another embodiment, there is provided a method of disinfecting a surface against Mupirocin-resistant bacteria contamination by applying to said surface a compound of the formula (I) or an acceptable salt or solvate thereof. The Mupirocin-resistant bacteria may be Mupirocin-resistant Staphylococcus aureus. In one embodiment, the method of disinfecting a surface is used for nasal decolonization. This may be applied topically to a subject prior to, for example, a surgery.

In one aspect, there is provided the use of a compound of formula (I) or a pharmaceutically acceptable salt or solvate thereof in the manufacture of a medicament for the treatment of a disease, disorder or condition selected from any bacterial infection, such as for instance a skin infection (e.g. boils, cuts, cellulitis, surgical wounds, impetigo), a blood infection, a respiratory disease (e.g. sinusitis, pneumonia), nasal decolonization, food poisoning or any other life-threatening systemic disease.

In one embodiment, there is provided the use of a compound of formula (I) or a pharmaceutically acceptable salt or solvate thereof in the manufacture of a medicament for the treatment of an acne condition. In one embodiment, the acne condition is due to a bacterial infection. The bacterial infection may be caused by Propionibacterium acnes.

In one aspect, there is provided the use of a compound of formula (I) or a pharmaceutically acceptable salt or solvate thereof for use in treating a bacterial infection.

In one aspect, there is provided the use of a compound of formula (I) or a pharmaceutically acceptable salt or solvate thereof in the manufacture of a medicament for the treatment of a bacterially caused disease, disorder or condition.

In certain embodiments, the medicament can be for treatment or for prophylactic use.

In one embodiment, the bacterial infection or bacterially caused disease, disorder or condition is caused by a Gram-positive bacteria.

In one embodiment, the bacterial infection or bacterially caused disease, disorder or condition is due to or caused by Staphylococcus aureus, Methicillin-resistant Staphylococcus aureus, Streptococcus pyogenes, Streptococcus pneumoniae or Propionibacterium acnes. In one embodiment, the bacterial infection or bacterially caused disease, disorder or condition is due to or caused by Staphylococcus aureus. In one embodiment, the bacterial infection or bacterially caused disease, disorder or condition is due to or caused by Methicillin-resistant Staphylococcus aureus. In one embodiment, the bacterial infection or bacterially caused disease, disorder or condition is due to or caused by a Methicillin-resistant Staphylococcus aureus strain that is resistant to Mupirocin. In one embodiment, the bacterial infection or bacterially caused disease, disorder or condition is due to or caused by a Methicillin-resistant Staphylococcus aureus strain that is resistant to Linezolid, Retapamulin or Tigecycline. In one embodiment, the bacterial infection or bacterially caused disease, disorder or condition is due to or caused by a Staphylococcus aureus strain that is resistant to Linezolid, Retapamulin or Tigecycline. In one embodiment, the bacterial infection or bacterially caused disease, disorder or condition is due to or caused by Propionibacterium acnes.

In one embodiment, the bacterial infection or bacterially caused disease, disorder or condition is caused by bacteria that have gained a resistance against the penicillin-type antibiotics, Vancomycin, Linezolid, Retapamulin, Tigecycline or Mupirocin. In one embodiment, the bacteria have gained a resistance to Mupirocin. In one embodiment, the penicillin-type antibiotic is Methicillin.

For administration to human patients, the total daily dose of a compound of the invention can be in the range of 0.5 to 2 grams, but is not limited to that range depending on the mode of administration. The total daily dose may be administered in single or divided doses, and may, at the physicians discretion, fall outside of this typical range.

Administration can be oral or parenteral (such as topical and ocular) or otherwise. In the pharmaceutical composition of the compounds of the invention excipients can be used. The term “excipient” encompasses diluents, carriers and adjuvants.

If the compounds are administered in tablets such as for example disclosed in Tablets, Vol. 1, by H. Liberman and L. Lachman (Marcel Dekker, New York, 1980).

The compounds of the invention may also be administered directly into the blood stream, into muscle or into an internal organ. Suitable means for parenteral administration include intravenous, intraarterial, intraperitoneal, intrathecal, intraventricular, intraureathral, intrasternal, intracranial, intramuscular, ocular and subcutaneous. Suitable devices for parenteral administration include needle injectors, needle free injectors and infusion techniques.

The compounds may also be administered topically to the skin or mucosa, that is, dermally, intranasal (such as a spray) or transdermal. In one embodiment, the compounds of formula (I) are especially useful in such topical applications where they can combat Methicillin-resistant Staphylococcus aureus strains.

The compounds of the invention may also be administered directly to the eye, nose or ear, typically in the form of drops of micronized suspension or solution in isotonic, pH-adjusted, sterile saline.

The compounds can also be inhaled to treat infection of the respiratory tract. Typical inhalers and inhalation formulations can be used. The formula (I) provides general definitions of the compounds according to the invention.

### EXAMPLES

In the examples described below, unless otherwise indicated, all temperatures in the following description are in degrees Celsius and all parts and percentages are by weight, unless indicated otherwise. Reagents useful for synthesizing compounds may be purchased from commercial suppliers, such as Merck Millipore, GL Biochem China, Creosalus Advanced Chemtech, Anaspec, Bachem, Chempep, Iris Biotech, Polypeptides, Sigma-Aldrich (Merck) and others, and used without further purification, unless otherwise indicated, or obtained or prepared according to techniques known in the art.

HPLC was conducted on a Shimadzu Prominence system. Mass spectrometry was conducted using a Shimadsu LC-MS system.

All the NMR experiments for ¹H (400.13 MHz) and ¹³C (100.61 MHz) nuclei were performed on a Bruker Ultrashield 400+ NMR spectrometer. NMR spectra are reported in ppm with reference to an internal tetramethylsilane standard (0.00 ppm for 1H and ¹³C) or solvent peak(s) of CD₃OD (3.31 and 49.0 ppm). When peak multiplicities are reported, the following abbreviations are used: s=singlet, d=doublet, t=triplet, q=quartet, m=multiplet, br=broadened, dd=doublet of doublets, dt=doublet of triplets, bs=broadened singlet. Coupling constants, when given, are reported in hertz.

### Example 1: Preparation of Compound 34

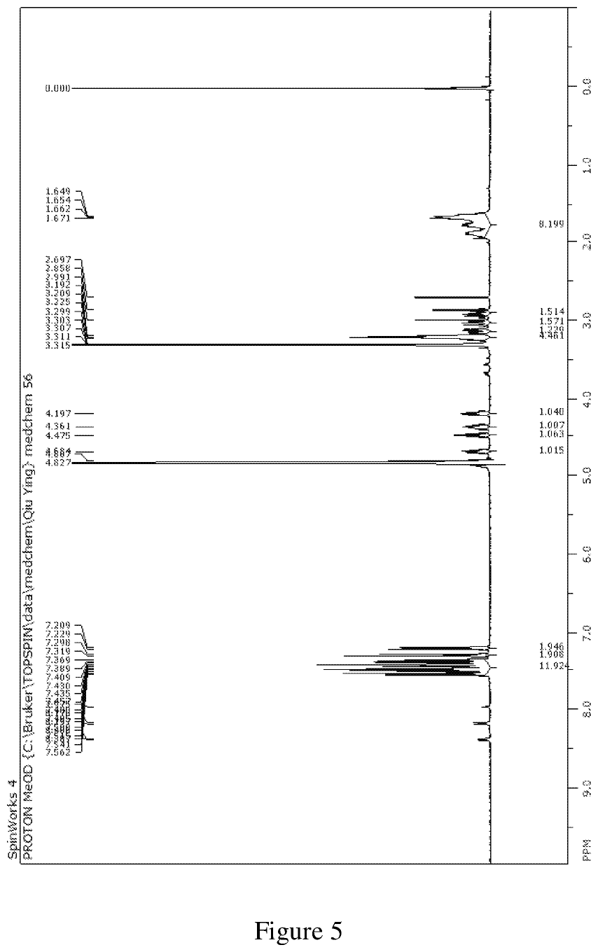

1. Swell Rink amide resin (1 mmol scale, 3.57 g, GL Biochem (China), Loading factor: 0.28) in piperidine:DMF (20% v/v) at RT for 1 hour.
2. Filter off excess solvent/reagents and wash resin with DMF (˜10 ml×2), CH₃OH (˜10 ml×2) followed by DMF (˜10 ml×2) again.
3. Dissolve Fmoc-D-Arg(Pbf)-OH (2 mmol, 2 eq, 1.3 g, Creosalus Advanced ChemTech), HBTU (2 mmol, 2 eq.), HOAt, DIPEA (2 mmol, 2 eq.) in DMF (20 mL) and allow this mixture to react with the resin with stirring at RT for 1 h.
4. Filter off excess solvent/reagents and wash resin with DMF (˜10 ml×2), CH₃OH (˜10 ml×2) followed by DMF (˜10 ml×2) again.
5. Introduce piperidine:DMF (20% v/v) into the resin with stirring at RT for 0.5 h.
6. Filter off excess solvent/reagents and wash resin with DMF (˜10 ml×2), CH₃OH (˜10 ml×2) followed by DMF (˜10 ml×2) again.
7. Dissolve N-Fmoc-4-(4-chlorophenyl)-D-phenylalanine (2 mmol, 2 eq, 1.0 g, Amatek Chemical, China), HBTU (2 mmol, 2 eq.), HOAt, DIPEA (2 mmol, 2 eq.) in DMF (20 mL) and allow this mixture to react with the resin with stirring at RT for 1 h.
8. Filter off excess solvent/reagents and wash resin with DMF (˜10 ml×2), CH₃OH (˜10 ml×2) followed by DMF (˜10 ml×2) again.
9. Introduce piperidine:DMF (20% v/v) into the resin with stirring at RT for 0.5 h.
10. Filter off excess solvent/reagents and wash resin with DMF (˜10 ml×2), CH₃OH (˜10 ml×2) followed by DMF (˜10 ml×2) again.
11. Couple subsequent amino acid using Steps 6-10.
12. Wash resin with DMF (˜10 ml×2), CH₃OH (˜10 ml×2) followed by CH₂Cl₂ (˜10 ml×2).
13. Add trifluoroacetic acid (TFA) (2 mL for 0.1 mmol scale) and deionized water (2 drops, ˜50 μL). Allow this mixture to react with the resin with stirring and microwave (400 W, 60° C., 20 min).
14. Excess TFA was blown off with a N₂ gas stream to yield the crude target as yellow oil.
15. Purify the yellow oil with C18 reverse-phase HPLC (1 mL concentrated HCl in 2 L H₂O) to obtain target as a white solid (349 mg, 36.6%). ESI-MS m/z [M+H]⁺ 844.4; [M+2H]²⁺÷2 422.7. Electrospray ionization-mass spectrum results is shown in FIG. 4. NMR results is shown in FIG. 5.

Some of the comparators are made under similar conditions as outlined in Example 1.

### Specific Example: Compound 34

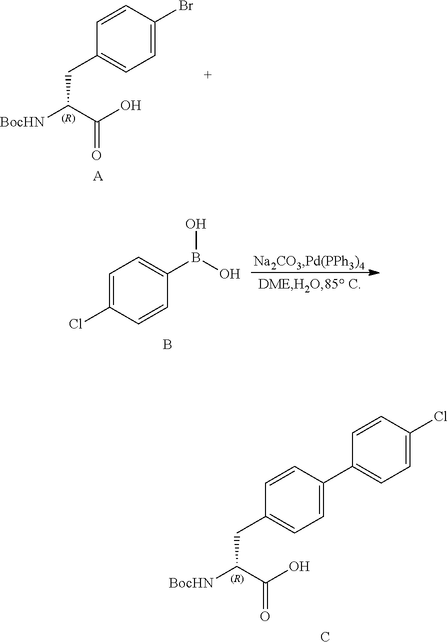

To a solution of A (170 g, 493.9 mmol) in DME (2040 mL) was added B (96.5 g, 617.3 mmol) followed by aq. Na₂CO₃ (2 M, 370.4 mL) and Pd(PPh₃)₄(17.1 g, 14.8 mmol). The mixture was stirred at 85° C. for 3 hrs. The reaction mixture was filtered and added EtOAc (3 L) and acidified by 1 M HCl to pH=5. The combined organic layer was washed with brine (1 L) and dried over anhydrous sodium sulfate, filtered and concentrated under reduced pressure. The obtained residue was purified by column chromatography on silica gel (100-200 mesh size) using petroleum ether/EtOAc (20:1-1:1) as eluent to give C (156 g, 332.0 mmol, 67.2% yield, 80.0% purity) as a white solid. 1H NMR 400 MHz, CDCl₃ δ ppm 7.54-7.76 (m, 1H) 7.43-7.53 (m, 5H) 7.36-7.42 (m, 2H) 7.23-7.30 (m, 2H) 4.37-4.76 (m, 1H) 2.90-3.32 (m, 2H) 1.27-1.47 (m, 9H) LCMS m/z [+ESI scan] 275.8, 397.8, 772.8.

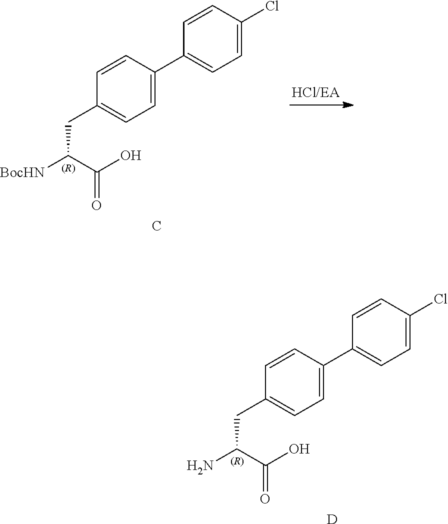

A mixture of C (125 g, 332.5 mmol) and HCl/EtOAc (4 M, 1.25 L) was stirred at 25° C. for 0.5 hr. The reaction mixture was filtrated and the resulting solid was dry by evaporating under vacuum to give D (85 g, 245.0 mmol, 73.6% yield, 90.0% purity, HCl) as a white solid. 1H NMR 400 MHz, DMSO-d6 δ ppm 13.83 (br s, 1H) 8.65 (br s, 3H) 7.59-7.73 (m, 4H) 7.50 (d, J=8.53 Hz, 2H) 7.40 (br d, J=8.03 Hz, 2H) 4.17 (br s, 1H) 3.23 (br d, J=5.52 Hz, 2H) LCMS m/z [+ESI scan] 276.0.

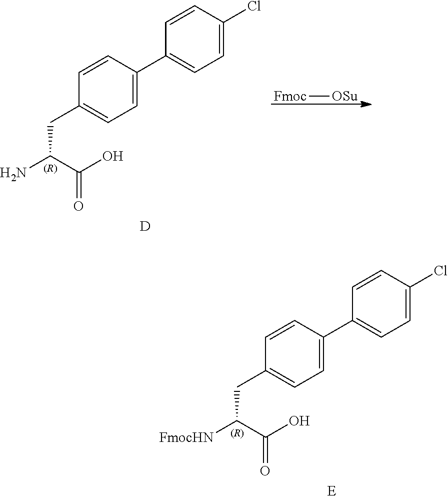

To a solution of D (60 g, 192.2 mmol, HCl) in H2O (1200 mL) and acetone (3600 mL) was added Fmoc-OSu (84.3 g, 249.8 mmol) and NaHCO₃(82.6 g, 984.1 mmol, 38.2 mL) until pH=8. The mixture was stirred at 25° C. for 16 hrs. The resulting mixture was concentrated in vacuo and the aqueous phase was added with 1.0 M HCl solution till the pH=4˜5, filtrated, the solid was washed with water (1.2 L×2), filtrated. The solid stirred in acetonitrile (3500 mL), filtrated, the resulting solid was dry by evaporating under vacuum to give E (82.3 g, 155.6 mmol, 80.9% yield, 94.18% purity) as a white solid. 1H NMR 400 MHz, DMSO-d6 δ ppm 7.87 (br d, J=7.53 Hz, 2H) 7.51-7.69 (m, 7H) 7.48 (d, J=8.53 Hz, 2H) 7.24-7.43 (m, 6H) 4.08-4.26 (m, 4H) 3.14 (dd, J=13.68, 4.14 Hz, 1H) 2.92 (dd, J=13.55, 10.29 Hz, 1H) LCMS m/z [+ESI scan] 178.8, 275.9, 497.9, 1017.1.

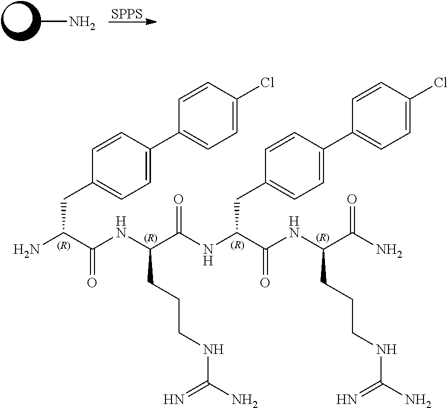

The peptide was synthesized using standard Fmoc chemistry. 1) Add DMF to the vessel containing MBHA Resin (sub: 0.6 mmol/g, 110.0 mmol, 183.0 g) and swell for 2 hours. 2) Add Fmoc-Rink linker and mix 30 seconds, then add activation buffer, N₂ bubbling for about 1 hour. 3) Drain and then DMF wash 30 sec with 3 times. 4) Add 20% piperidine/DMF and mix for 30 min. 5) Drain and then DMF wash 30 sec with 5 times. 6) Add Fmoc-amino acid solution and mix 30 seconds, then add activation buffer, N₂ bubbling for about 1 hour. 7) Repeat step 3 to 6 for next amino acid coupling. Note:

| # | Materials | Coupling reagents |
|---|---|---|
| 1 | Fmoc-Rink linker (2.0 eq) | HBTU (1.9 eq) and |
| | | DIEA (4.0 eq) |
| 2 | Fmoc-D-Arg(pbf)-OH (2.0 eq) | HBTU (1.9 eq) and |
| | | DIEA (4.0 eq) |
| 3 | (R)-2-((((9H-fluoren-9-yl)methoxy)carbonyl) | HATU (1.9 eq) and |
| | amino)-3-(4′-chloro-[1,1′-biphenyl-4-yl) | DIEA (4.0 eq) |
| | propanoic acid (2.0 eq) | |
| 4 | Fmoc-D-Arg(pbf)-OH (2.0 eq) | HATU (1.9 eq) and |
| | | DIEA (4.0 eq) |
| 5 | (R)-2-((((9H-fluoren-9-yl)methoxy)carbonyl) | HATU (1.9 eq) and |
| | amino)-3-(4′-chloro-[1,1′-biphenyl-4-yl) | DIEA (4.0 eq) |
| | propanoic acid (2.0 eq) | |

20% piperidine in DMF was used for Fmoc deprotection for 30 min. The coupling reaction was monitored by ninhydrin test, and the resin was washed with DMF for 5 times.
Peptide Cleavage and Purification: 1) Add cleavage buffer (95% TFA/2.5% Thioanisole/2.5% H₂O) to the flask containing the side chain protected peptide at room temperature and stir for 3 hours. 2) The peptide is precipitated with cold tert-butyl methyl ether and centrifuged (2 min at 5000 rpm). 3) Tert-butyl methyl ether washes two additional times. 4) Dry the crude peptide under vacuum 2 hours. 5) Purify the crude peptide by Pre_HPLC (A: 0.005 mol/L HCl in H₂O, B: ACN) to give the final product (51.2 g, 99.33% purity, 55.1% yield). 6) Purification conditions:

Separation condition

| | |
|---|---|
| Dissolution condition | Dissolve in DMF/H₂O |
| Instrument | Gilson GX-215 |
| Mobile Phase | A: H₂O (0.005 mol/L HCl in H₂O) |
| | B: CH₃CN |
| Gradient | 10-50%-60 min. Retention time: 35 min |
| Column | Luna25*200 mm, C18 10 um, |
| | 110A + Gemin150*30 mm, C18 5 um, 110A |
| Flow Rate | 20 mL/Min |
| Wavelength | 214/254 nm |
| Oven Tem. | Room temperature |

LCMS m/z [+ESI scan] 258.1, 422.7, 844.2.

The examples of the present invention are made under similar conditions as outlined in Example 1. Table 1 shows the ESI-MS (m/z) data for the exemplary compounds.

TABLE 1
ESI-MS data of compounds

| | ESI-MS | |
|---|---|---|
| Compound | [M + H]⁺ | [M + 2H]² ÷ 2 |
| 15 | 811.0 | 406.2 |
| 16 | 811.0 | 406.2 |
| 17 | 844.4 | 423.1 |
| 18 | 844.0 | 423.5 |
| 19 | 856.9 | 428.9 |
| 20 | 856.9 | 428.4 |
| 27 | 811.0 | 406.0 |
| 28 | 811.0 | 406.2 |
| 33 | 844.4 | 422.8 |
| 35 | 688.3 | not detected |
| 39 | Not detected | 501.0 |
| 41 | 802.4 | not detected |
| 42 | Not detected | 423.1 |
| 43 | Not detected | 422.9 |
| 44 | Not detected | 423.3 |
| 45 | Not detected | 423.8 |

### Example 2: Biological Activity Measurement

The MICs of test compounds were determined using the microdilution method from the Clinical and Laboratory Standards Institute (CLSI) guidelines (Clinical and Laboratory Standards Institute. Methods for dilution antimicrobial susceptibility tests for bacteria that grow aerobically; approved standard—ninth edition. Document M07-A9. Vol. 32 No. 2. Wayne, Pa.: CLSI; 2012). Briefly, bacteria were grown fresh from frozen stock in Mueller Hinton 2 (MH2) agar at 37° C. After an overnight incubation, 5 bacteria colonies were selected to grow in cation-adjusted MH2 broth in a shaking incubator (140 RPM) at 37° C. Cells were grown to an optical density (OD₆₀₀) of 0.15-0.16 determined using a spectrophotometer (Molecular Devices Spectra Max Plus), which corresponds to ˜1×10⁸ CFU/mL. Test compounds were constituted into 4 mM DMSO stock solutions and then subjected to 2-fold serial dilution in a 96-well plate with concentrations ranging from 100 to 0.195 μM in duplicates. 50 mL of microbial culture containing ˜1×10⁶ CFU/mL of microbes in the respective broths was introduced into each well containing 50 μL of compound solution. After an overnight incubation at 35° C., (220 RPM), OD₆₀₀ measurements were conducted using the microplate spectrophotometer. The MIC was defined as the lowest antibiotic or compound concentration (μM) required to inhibit bacteria growth.

The 11th well was used as the growth control well (medium with bacterial inoculums, no antibacterial) while the 12th well was the sterility control well (medium only). Table 2 illustrates a typical sample layout.

TABLE 2
Typical plate layout for the setup of a 96-well plate for the cell-based assay

| | Conc. | | | | | | | | | | | | |
|---|---|---|---|---|---|---|---|---|---|---|---|---|---|
| | (μM) | 1 | 2 | 3 | 4 | 5 | 6 | 7 | 8 | 9 | 10 | 11 | 12 |
| A | Linezolid | 100 | 50 | 25 | 12.5 | 6.25 | 3.13 | 1.56 | 0.78 | 0.39 | 0.20 | GC | SC |
| B | Linezolid | 100 | 50 | 25 | 12.5 | 6.25 | 3.13 | 1.56 | 0.78 | 0.39 | 0.20 | GC | SC |
| C | | | | | | | | | | | | GC | SC |
| D | | | | | | | | | | | | GC | SC |
| E | | | | | | | | | | | | GC | SC |
| F | | | | | | | | | | | | | |
| G | | | | | | | | | | | | | |
| H | | | | | | | | | | | | GC | SC |

Table 3 shows the structures of examples and comparators and their corresponding minimum inhibitory concentration (MIC) against ATCC-BAA-1556.

Table 4 compares the minimum inhibitory concentrations of Compounds 33 and 34 against commercial antibacterial compounds Linezolid, Mupirocin, Retapamulin, Vancomycin as well as Compound 2 (which is a comparator) in Mupirocin-resistant and Mupirocin-susceptible MRSA strains. Compounds 33 and 34 are shown to have about 4 fold improvements of bioactivity (in terms of lower MIC values) against Mupirocin-resistant MRSA strains as compared to Compound 2. For example, Compounds 33 and 34 each have a MIC value of 3.125 μM against ATCC-BAA-1556 whereas Compound 2 has a MIC value of 12.5 μM against the same strain.

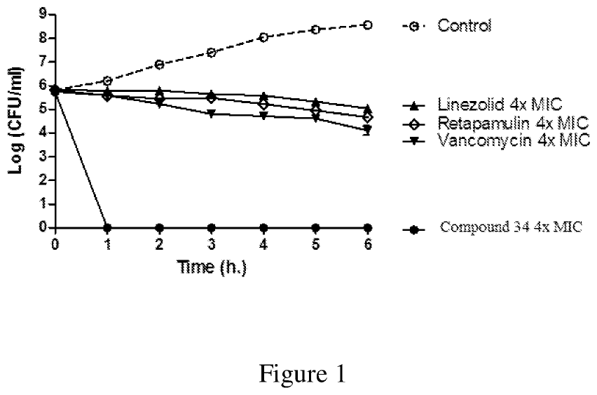

A time-kill assay was performed (FIG. 1) and Compound 34 is shown to kill Mupirocin-resistant MRSA (ATCC-BAA-1556) more rapidly as compared to Linezolid, Retapamulin and Vancomycin at 4×MIC.

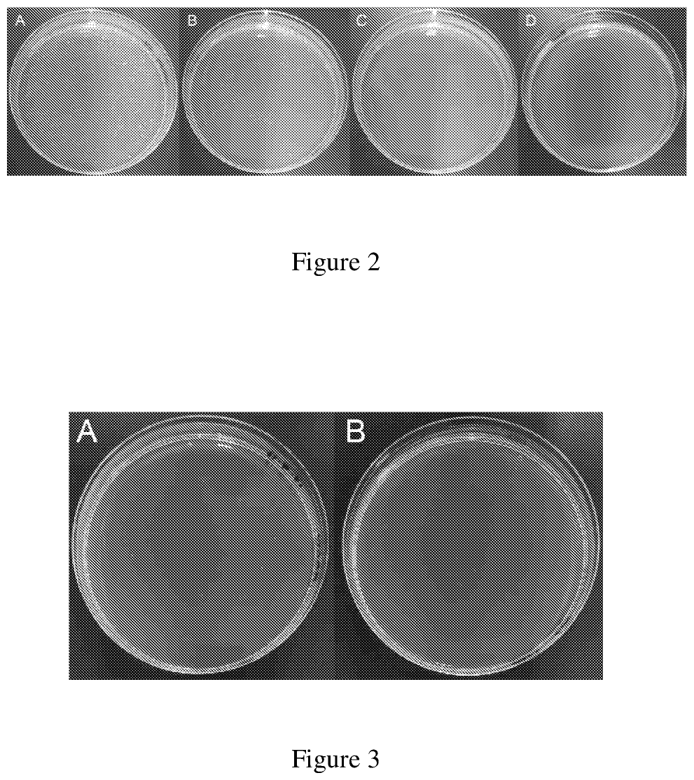

FIG. 2 shows a bacterial/static determination assay where Compound 34 is shown to have higher bactericidal activity than Linezolid, Retapamulin and Vancomycin against Mupirocin-resistant MRSA (ATCC-BAA-1556) at 4×MIC.

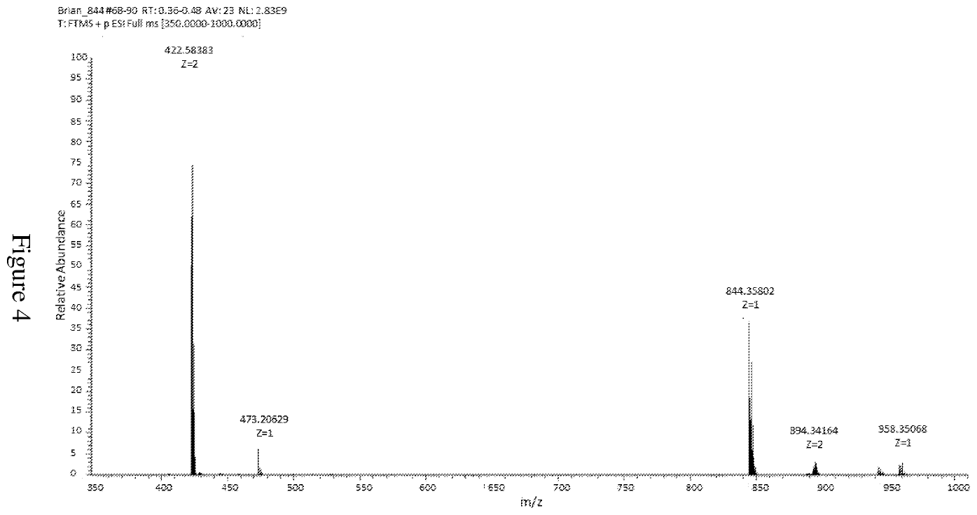

FIG. 3 shows the determination of the minimum bactericidal concentration of Compound 34 using Mupirocin-resistant MRSA at MIC and 2×MIC.

TABLE 3
Structures and MICs (Mupirocin-resistant MRSA ATCC-BAA-1556)

| Compound | | Comparator/ | MIC |
|---|---|---|---|
| No. | Structure | Example | (μM) |
| 1 | 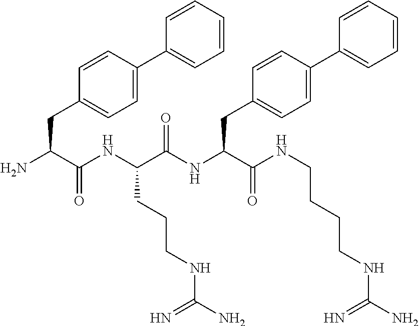 [SMILES: N=C(N)NCCCCNC(=O)[C@H](Cc1ccc(-c2ccccc2)cc1)NC(=O)[C@H](CCCNC(=N)N)NC(=O)[C@@H](N)Cc1ccc(-c2ccccc2)cc1] | Comparator | 12.5 |
| 2 | 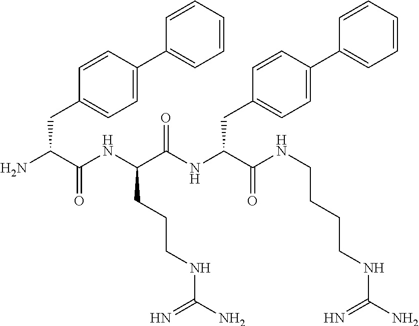 [SMILES: N=C(N)NCCCCNC(=O)[C@@H](Cc1ccc(-c2ccccc2)cc1)NC(=O)[C@@H](CCCNC(=N)N)NC(=O)[C@H](N)Cc1ccc(-c2ccccc2)cc1] | Comparator | 12.5 |
| 3 | 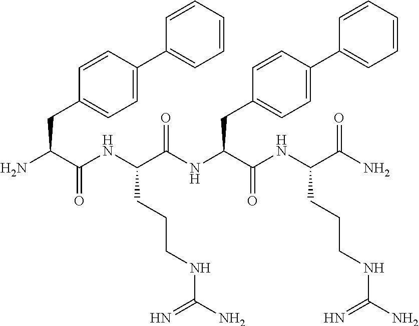 [SMILES: N=C(N)NCCC[C@H](NC(=O)[C@H](Cc1ccc(-c2ccccc2)cc1)NC(=O)[C@H](CCCNC(=N)N)NC(=O)[C@@H](N)Cc1ccc(-c2ccccc2)cc1)C(N)=O] | Comparator | 12.5 |
| 4 | 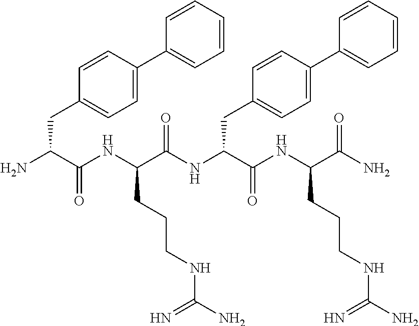 [SMILES: N=C(N)NCCC[C@@H](NC(=O)[C@@H](Cc1ccc(-c2ccccc2)cc1)NC(=O)[C@@H](CCCNC(=N)N)NC(=O)[C@H](N)Cc1ccc(-c2ccccc2)cc1)C(N)=O] | Comparator | 12.5 |
| 5 | 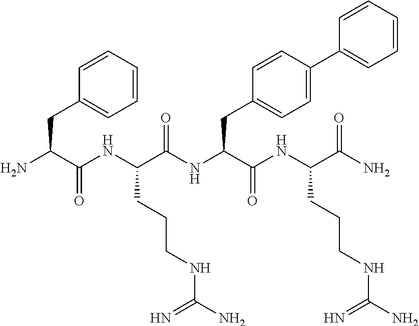 [SMILES: N=C(N)NCCC[C@H](NC(=O)[C@H](Cc1ccc(-c2ccccc2)cc1)NC(=O)[C@H](CCCNC(=N)N)NC(=O)[C@@H](N)Cc1ccccc1)C(N)=O] | Comparator | >100 |
| 6 | 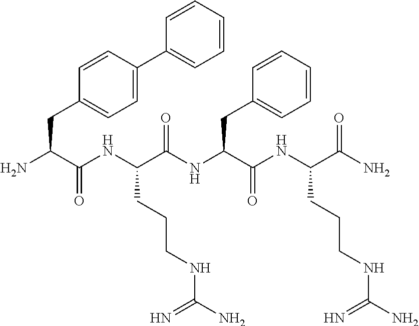 [SMILES: N=C(N)NCCC[C@H](NC(=O)[C@H](Cc1ccccc1)NC(=O)[C@H](CCCNC(=N)N)NC(=O)[C@@H](N)Cc1ccc(-c2ccccc2)cc1)C(N)=O] | Comparator | >100 |
| 7 | 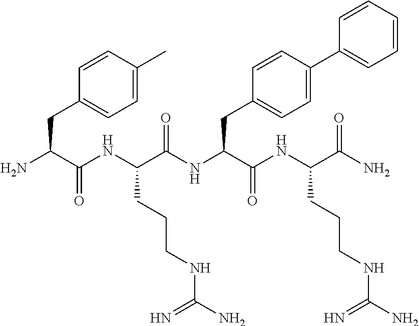 [SMILES: Cc1ccc(C[C@H](N)C(=O)N[C@@H](CCCNC(=N)N)C(=O)N[C@@H](Cc2ccc(-c3ccccc3)cc2)C(=O)N[C@@H](CCCNC(=N)N)C(N)=O)cc1] | Comparator | 100 |
| 8 | 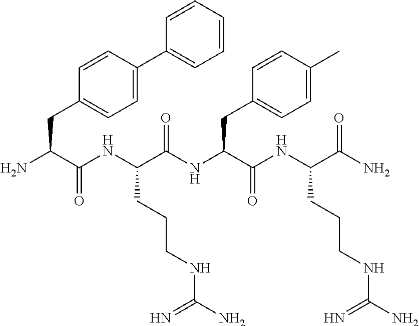 [SMILES: Cc1ccc(C[C@H](NC(=O)[C@H](CCCNC(=N)N)NC(=O)[C@@H](N)Cc2ccc(-c3ccccc3)cc2)C(=O)N[C@@H](CCCNC(=N)N)C(N)=O)cc1] | Comparator | >100 |
| 9 | 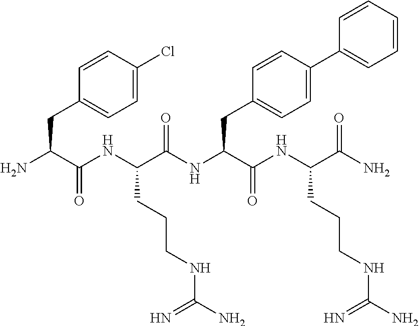 [SMILES: N=C(N)NCCC[C@H](NC(=O)[C@H](Cc1ccc(-c2ccccc2)cc1)NC(=O)[C@H](CCCNC(=N)N)NC(=O)[C@@H](N)Cc1ccc(Cl)cc1)C(N)=O] | Comparator | 100 |
| 10 | 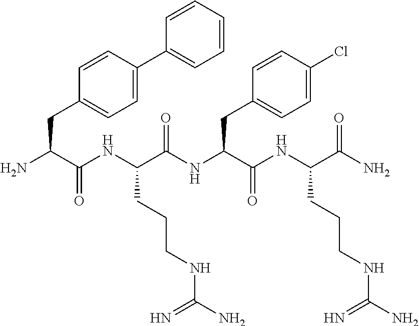 [SMILES: N=C(N)NCCC[C@H](NC(=O)[C@H](Cc1ccc(Cl)cc1)NC(=O)[C@H](CCCNC(=N)N)NC(=O)[C@@H](N)Cc1ccc(-c2ccccc2)cc1)C(N)=O] | Comparator | 100 |
| 11 | 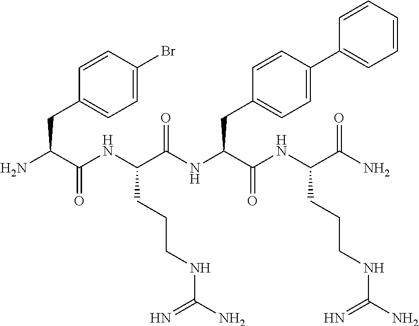 [SMILES: N=C(N)NCCC[C@H](NC(=O)[C@H](Cc1ccc(-c2ccccc2)cc1)NC(=O)[C@H](CCCNC(=N)N)NC(=O)[C@@H](N)Cc1ccc(Br)cc1)C(N)=O] | Comparator | 50 |
| 12 | 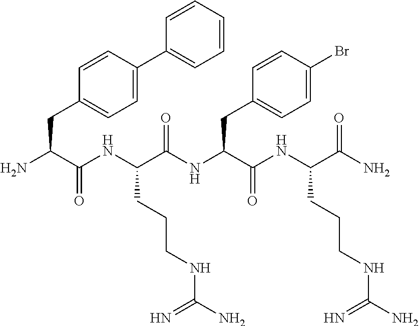 [SMILES: N=C(N)NCCC[C@H](NC(=O)[C@H](Cc1ccc(Br)cc1)NC(=O)[C@H](CCCNC(=N)N)NC(=O)[C@@H](N)Cc1ccc(-c2ccccc2)cc1)C(N)=O] | Comparator | 100 |
| 13 | 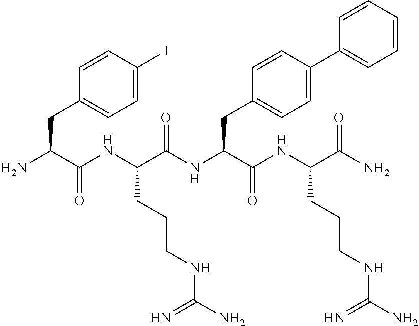 [SMILES: N=C(N)NCCC[C@H](NC(=O)[C@H](Cc1ccc(-c2ccccc2)cc1)NC(=O)[C@H](CCCNC(=N)N)NC(=O)[C@@H](N)Cc1ccc(I)cc1)C(N)=O] | Comparator | 50 |
| 14 | 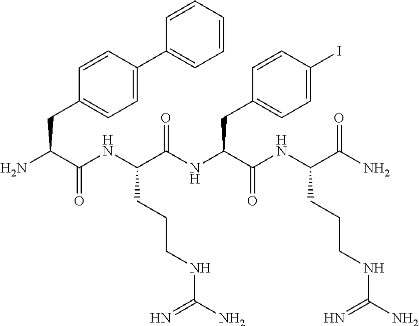 [SMILES: N=C(N)NCCC[C@H](NC(=O)[C@H](Cc1ccc(I)cc1)NC(=O)[C@H](CCCNC(=N)N)NC(=O)[C@@H](N)Cc1ccc(-c2ccccc2)cc1)C(N)=O] | Comparator | 100 |
| 15 | 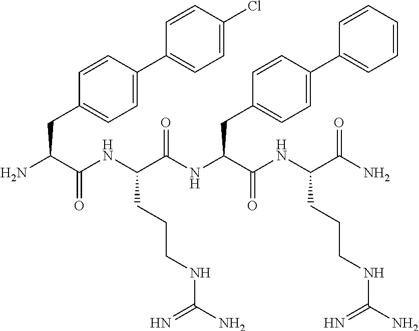 [SMILES: N=C(N)NCCC[C@H](NC(=O)[C@H](Cc1ccc(-c2ccccc2)cc1)NC(=O)[C@H](CCCNC(=N)N)NC(=O)[C@@H](N)Cc1ccc(-c2ccc(Cl)cc2)cc1)C(N)=O] | Example | 6.25 |
| 16 | 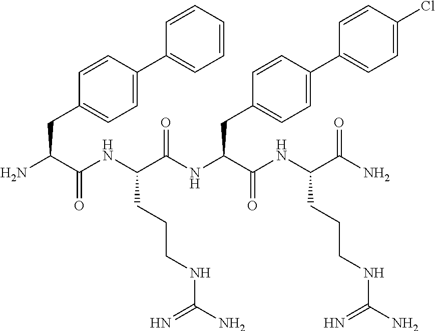 [SMILES: N=C(N)NCCC[C@H](NC(=O)[C@H](Cc1ccc(-c2ccc(Cl)cc2)cc1)NC(=O)[C@H](CCCNC(=N)N)NC(=O)[C@@H](N)Cc1ccc(-c2ccccc2)cc1)C(N)=O] | Example | 6.25 |
| 17 | 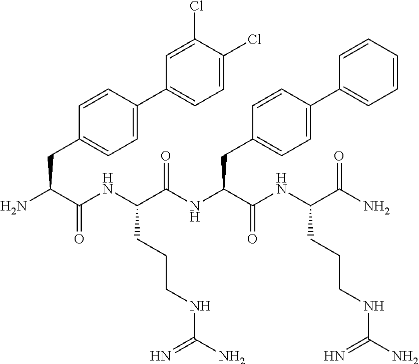 [SMILES: N=C(N)NCCC[C@H](NC(=O)[C@H](Cc1ccc(-c2ccccc2)cc1)NC(=O)[C@H](CCCNC(=N)N)NC(=O)[C@@H](N)Cc1ccc(-c2ccc(Cl)c(Cl)c2)cc1)C(N)=O] | Example | 6.25 |
| 18 | 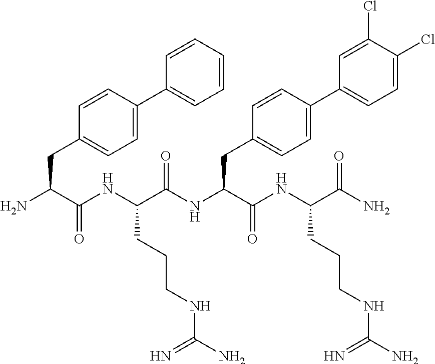 [SMILES: N=C(N)NCCC[C@H](NC(=O)[C@H](Cc1ccc(-c2ccc(Cl)c(Cl)c2)cc1)NC(=O)[C@H](CCCNC(=N)N)NC(=O)[C@@H](N)Cc1ccc(-c2ccccc2)cc1)C(N)=O] | Example | 6.25 |
| 19 | 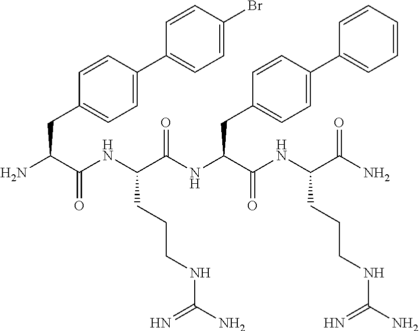 [SMILES: N=C(N)NCCC[C@H](NC(=O)[C@H](Cc1ccc(-c2ccccc2)cc1)NC(=O)[C@H](CCCNC(=N)N)NC(=O)[C@@H](N)Cc1ccc(-c2ccc(Br)cc2)cc1)C(N)=O] | Example | 6.25 |
| 20 | 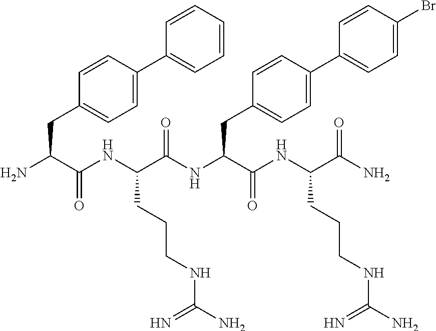 [SMILES: N=C(N)NCCC[C@H](NC(=O)[C@H](Cc1ccc(-c2ccc(Br)cc2)cc1)NC(=O)[C@H](CCCNC(=N)N)NC(=O)[C@@H](N)Cc1ccc(-c2ccccc2)cc1)C(N)=O] | Example | 6.25 |
| 21 | 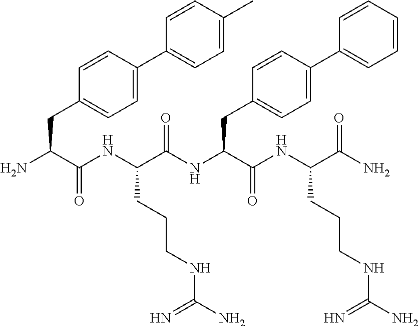 [SMILES: Cc1ccc(-c2ccc(C[C@H](N)C(=O)N[C@@H](CCCNC(=N)N)C(=O)N[C@@H](Cc3ccc(-c4ccccc4)cc3)C(=O)N[C@@H](CCCNC(=N)N)C(N)=O)cc2)cc1] | Comparator | 12.5 |
| 22 | 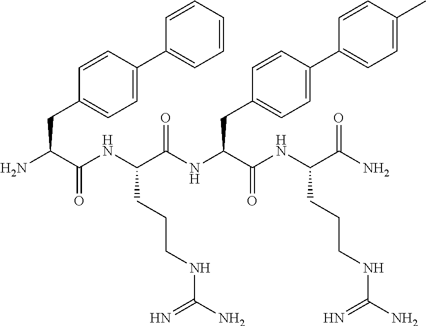 [SMILES: Cc1ccc(-c2ccc(C[C@H](NC(=O)[C@H](CCCNC(=N)N)NC(=O)[C@@H](N)Cc3ccc(-c4ccccc4)cc3)C(=O)N[C@@H](CCCNC(=N)N)C(N)=O)cc2)cc1] | Comparator | 12.5 |
| 23 | 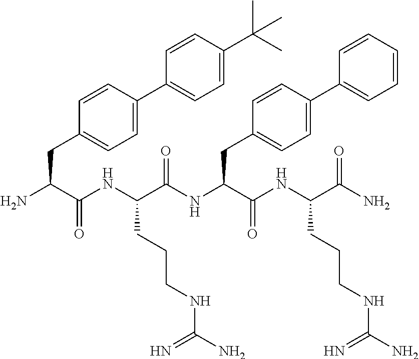 [SMILES: CC(C)(C)c1ccc(-c2ccc(C[C@H](N)C(=O)N[C@@H](CCCNC(=N)N)C(=O)N[C@@H](Cc3ccc(-c4ccccc4)cc3)C(=O)N[C@@H](CCCNC(=N)N)C(N)=O)cc2)cc1] | Comparator | 12.5 |
| 24 | 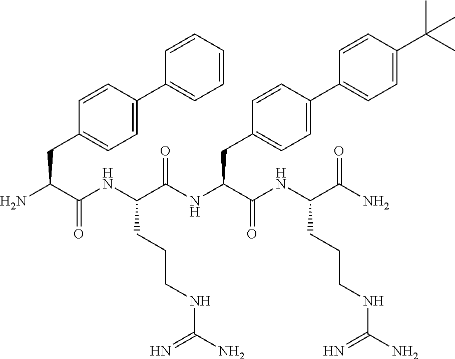 [SMILES: CC(C)(C)c1ccc(-c2ccc(C[C@H](NC(=O)[C@H](CCCNC(=N)N)NC(=O)[C@@H](N)Cc3ccc(-c4ccccc4)cc3)C(=O)N[C@@H](CCCNC(=N)N)C(N)=O)cc2)cc1] | Comparator | 12.5 |
| 25 | 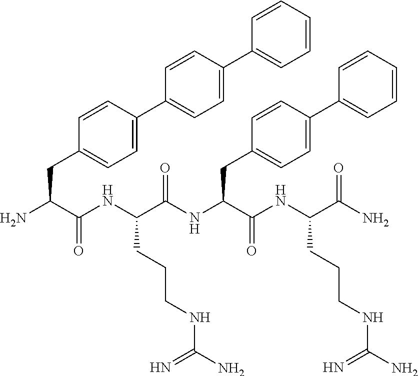 [SMILES: N=C(N)NCCC[C@H](NC(=O)[C@H](Cc1ccc(-c2ccccc2)cc1)NC(=O)[C@H](CCCNC(=N)N)NC(=O)[C@@H](N)Cc1ccc(-c2ccc(-c3ccccc3)cc2)cc1)C(N)=O] | Comparator | 100 |
| 26 | 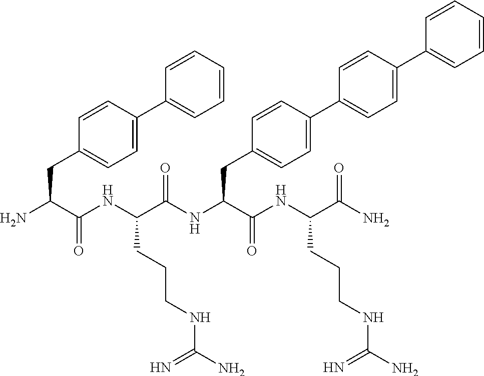 [SMILES: N=C(N)NCCC[C@H](NC(=O)[C@H](Cc1ccc(-c2ccc(-c3ccccc3)cc2)cc1)NC(=O)[C@H](CCCNC(=N)N)NC(=O)[C@@H](N)Cc1ccc(-c2ccccc2)cc1)C(N)=O] | Comparator | 25 |
| 27 | 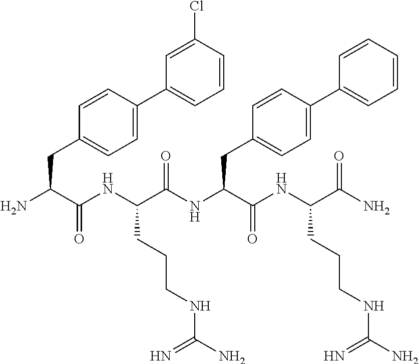 [SMILES: N=C(N)NCCC[C@H](NC(=O)[C@H](Cc1ccc(-c2ccccc2)cc1)NC(=O)[C@H](CCCNC(=N)N)NC(=O)[C@@H](N)Cc1ccc(-c2cccc(Cl)c2)cc1)C(N)=O] | Example | 6.25 |
| 28 | 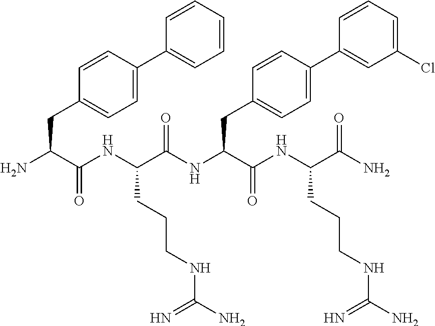 [SMILES: N=C(N)NCCC[C@H](NC(=O)[C@H](Cc1ccc(-c2cccc(Cl)c2)cc1)NC(=O)[C@H](CCCNC(=N)N)NC(=O)[C@@H](N)Cc1ccc(-c2ccccc2)cc1)C(N)=O] | Example | 6.25 |
| 29 | 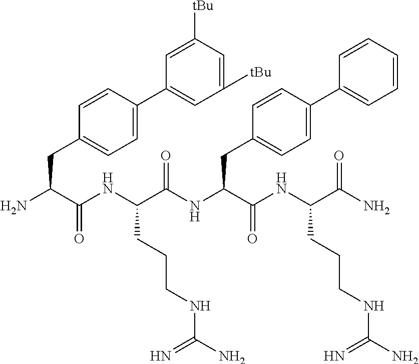 [SMILES: CC(C)(C)c1cc(-c2ccc(C[C@H](N)C(=O)N[C@@H](CCCNC(=N)N)C(=O)N[C@@H](Cc3ccc(-c4ccccc4)cc3)C(=O)N[C@@H](CCCNC(=N)N)C(N)=O)cc2)cc(C(C)(C)C)c1] | Comparator | 50 |
| 30 | 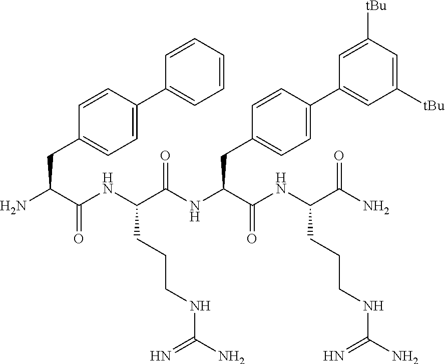 [SMILES: CC(C)(C)c1cc(-c2ccc(C[C@H](NC(=O)[C@H](CCCNC(=N)N)NC(=O)[C@@H](N)Cc3ccc(-c4ccccc4)cc3)C(=O)N[C@@H](CCCNC(=N)N)C(N)=O)cc2)cc(C(C)(C)C)c1] | Comparator | 50 |
| 31 | 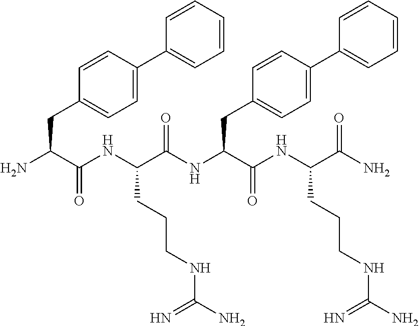 [SMILES: N=C(N)NCCC[C@H](NC(=O)[C@H](Cc1ccc(-c2ccccc2)cc1)NC(=O)[C@H](CCCNC(=N)N)NC(=O)[C@@H](N)Cc1ccc(-c2ccccc2)cc1)C(N)=O] | Comparator | 100 |
| 32 | 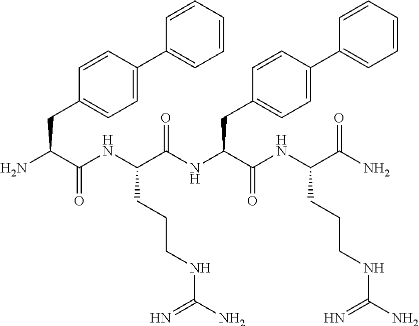 [SMILES: N=C(N)NCCC[C@H](NC(=O)[C@H](Cc1ccc(-c2ccccc2)cc1)NC(=O)[C@H](CCCNC(=N)N)NC(=O)[C@@H](N)Cc1ccc(-c2ccccc2)cc1)C(N)=O] | Comparator | 50 |
| 33 | 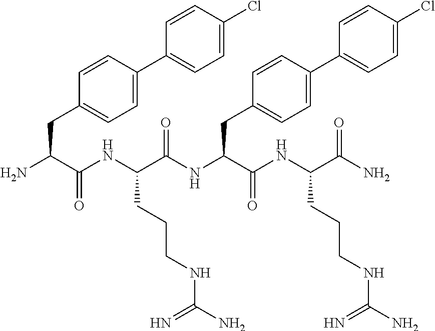 [SMILES: N=C(N)NCCC[C@H](NC(=O)[C@H](Cc1ccc(-c2ccc(Cl)cc2)cc1)NC(=O)[C@H](CCCNC(=N)N)NC(=O)[C@@H](N)Cc1ccc(-c2ccc(Cl)cc2)cc1)C(N)=O] | Example | 3.125 |
| 34 | 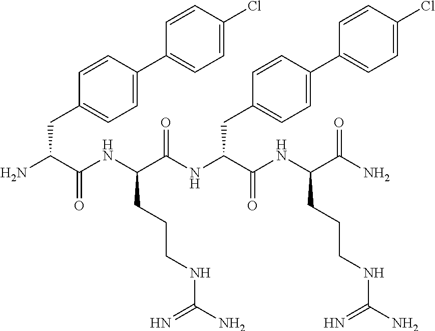 [SMILES: N=C(N)NCCC[C@@H](NC(=O)[C@@H](Cc1ccc(-c2ccc(Cl)cc2)cc1)NC(=O)[C@@H](CCCNC(=N)N)NC(=O)[C@H](N)Cc1ccc(-c2ccc(Cl)cc2)cc1)C(N)=O] | Example | 3.125 |
| 35 | 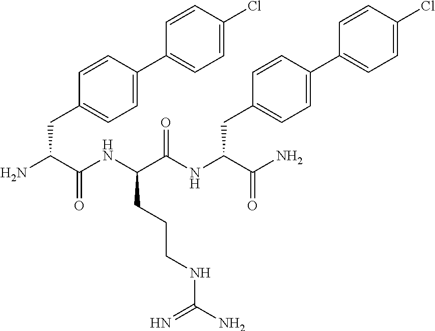 [SMILES: N=C(N)NCCC[C@@H](NC(=O)[C@H](N)Cc1ccc(-c2ccc(Cl)cc2)cc1)C(=O)N[C@H](Cc1ccc(-c2ccc(Cl)cc2)cc1)C(N)=O] | Example | 6.25 |
| 36 | 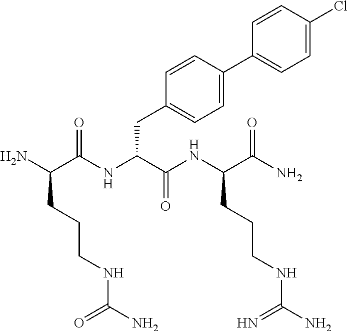 [SMILES: N=C(N)NCCC[C@@H](NC(=O)[C@@H](Cc1ccc(-c2ccc(Cl)cc2)cc1)NC(=O)[C@H](N)CCCNC(N)=O)C(N)=O] | Comparator | >100 |
| 37 | 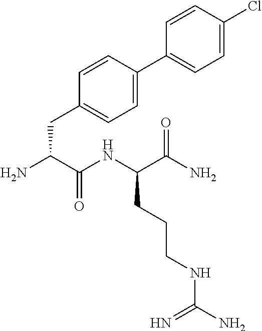 [SMILES: N=C(N)NCCC[C@@H](NC(=O)[C@H](N)Cc1ccc(-c2ccc(Cl)cc2)cc1)C(N)=O] | Comparator | >100 |
| 38 | 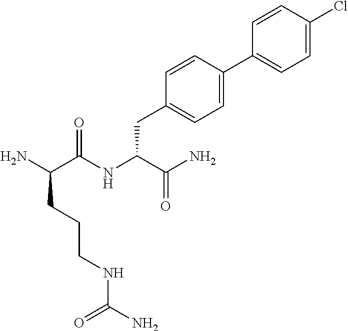 [SMILES: NC(=O)NCCC[C@@H](N)C(=O)N[C@H](Cc1ccc(-c2ccc(Cl)cc2)cc1)C(N)=O] | Comparator | >100 |
| 39 | 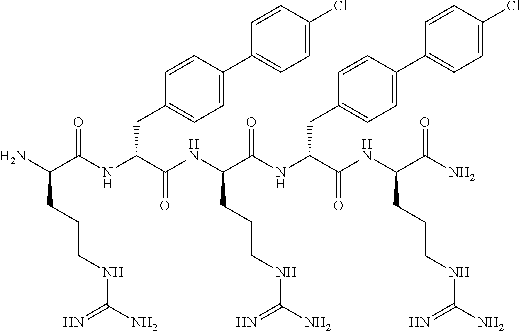 [SMILES: N=C(N)NCCC[C@@H](N)C(=O)N[C@H](Cc1ccc(-c2ccc(Cl)cc2)cc1)C(=O)N[C@H](CCCNC(=N)N)C(=O)N[C@H](Cc1ccc(-c2ccc(Cl)cc2)cc1)C(=O)N[C@H](CCCNC(=N)N)C(N)=O] | Example | 3.125 |
| 40 | 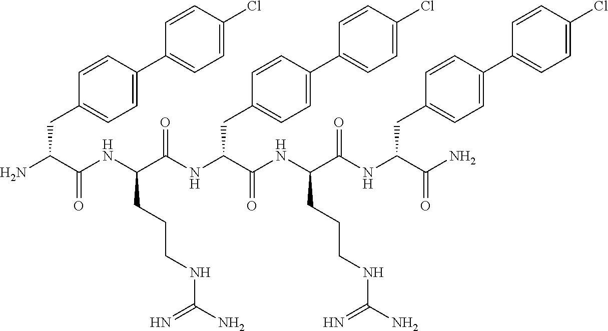 [SMILES: N=C(N)NCCC[C@@H](NC(=O)[C@H](N)Cc1ccc(-c2ccc(Cl)cc2)cc1)C(=O)N[C@H](Cc1ccc(-c2ccc(Cl)cc2)cc1)C(=O)N[C@H](CCCNC(=N)N)C(=O)N[C@H](Cc1ccc(-c2ccc(Cl)cc2)cc1)C(N)=O] | Comparator | >100 |
| 41 | 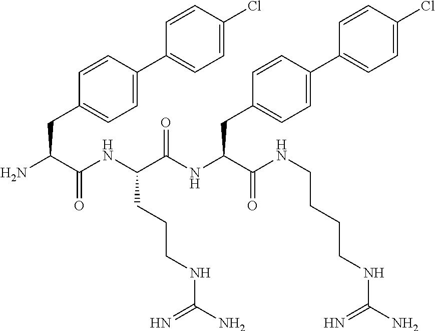 [SMILES: N=C(N)NCCCCNC(=O)[C@H](Cc1ccc(-c2ccc(Cl)cc2)cc1)NC(=O)[C@H](CCCNC(=N)N)NC(=O)[C@@H](N)Cc1ccc(-c2ccc(Cl)cc2)cc1] | Example | 6.25 |
| 42 | 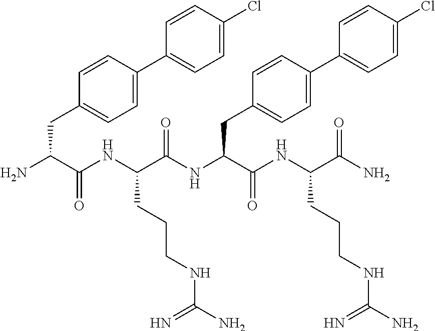 [SMILES: N=C(N)NCCC[C@H](NC(=O)[C@H](Cc1ccc(-c2ccc(Cl)cc2)cc1)NC(=O)[C@H](CCCNC(=N)N)NC(=O)[C@H](N)Cc1ccc(-c2ccc(Cl)cc2)cc1)C(N)=O] | Example | 6.25 |
| 43 | 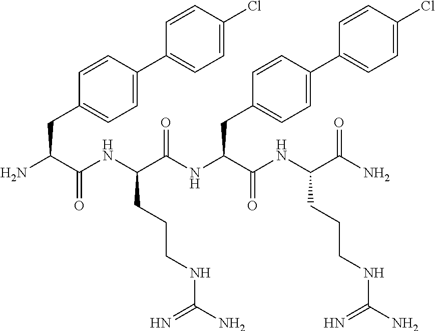 [SMILES: N=C(N)NCCC[C@H](NC(=O)[C@H](Cc1ccc(-c2ccc(Cl)cc2)cc1)NC(=O)[C@@H](CCCNC(=N)N)NC(=O)[C@@H](N)Cc1ccc(-c2ccc(Cl)cc2)cc1)C(N)=O] | Example | 6.25 |
| 44 | 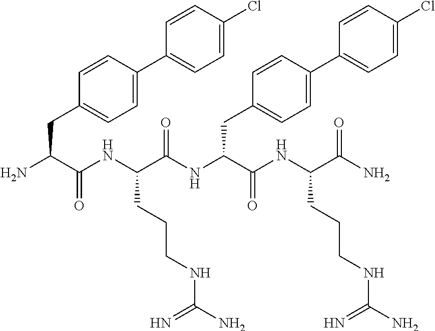 [SMILES: N=C(N)NCCC[C@H](NC(=O)[C@@H](Cc1ccc(-c2ccc(Cl)cc2)cc1)NC(=O)[C@H](CCCNC(=N)N)NC(=O)[C@@H](N)Cc1ccc(-c2ccc(Cl)cc2)cc1)C(N)=O] | Example | 6.25 |
| 45 | 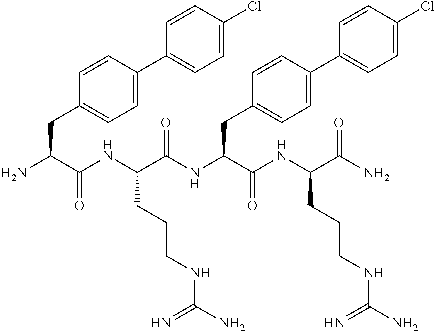 [SMILES: N=C(N)NCCC[C@H](NC(=O)[C@@H](N)Cc1ccc(-c2ccc(Cl)cc2)cc1)C(=O)N[C@@H](Cc1ccc(-c2ccc(Cl)cc2)cc1)C(=O)N[C@H](CCCNC(=N)N)C(N)=O] | Example | 6.25 |

TABLE 4
MICs (μM) of compounds against various MRSA strains

| | | | | | Compound | Compound | Compound |
|---|---|---|---|---|---|---|---|
| ATCC- | Linezolid | Mupirocin | Retapamulin | Vancomycin | 2 | 33 | 34 |

Mupirocin-resistant MRSA

| | | | | | | | |
|---|---|---|---|---|---|---|---|
| BAA-1556 | 6.25 | >100 | ≤0.2 | 0.78 | 12.5 | 3.125 | 3.125 |
| BAA-1708 | 6.25 | >100 | ≤0.2 | 0.78 | 6.25 | 1.56 | 1.56 |
| BAA-1750 | 6.25 | 25 | ≤0.2 | 0.78 | 6.25 | 1.56 | 1.56 |
| (USA200) | | | | | | | |

Mupirocin-susceptible MRSA

| | | | | | | | |
|---|---|---|---|---|---|---|---|
| BAA-1681 | 6.25 | ≤0.2 | ≤0.2 | 1.56 | 6.25 | 3.125 | 3.125 |
| (USA100) | | | | | | | |
| BAA-1756 | 6.25 | ≤0.2 | ≤0.2 | 0.78 | 6.25 | 3.125 | 1.56 |
| (USA300) | | | | | | | |
| BAA-1707 | 6.25 | ≤0.2 | ≤0.2 | 0.78 | 6.25 | 3.125 | 3.125 |
| (USA400) | | | | | | | |
| BAA-2762 | 6.25 | ≤0.2 | ≤0.2 | 0.78 | 6.25 | 3.125 | 3.125 |
| (EMRSA15, | | | | | | | |
| ST22) | | | | | | | |
| BAA-1754 | 6.25 | ≤0.2 | ≤0.2 | 0.78 | 6.25 | 3.125 | 3.125 |
| (ST45) | | | | | | | |
| 33592 | 6.25 | ≤0.2 | ≤0.2 | 0.78 | 6.25 | 3.125 | 3.125 |
| (ST239) | | | | | | | |
| 700699 | 3.125 | ≤0.2 | ≤0.2 | 6.25 | 6.25 | 3.125 | 3.125 |
| (VISA) | | | | | | | |

### Example 3: Activity of Compound 34 in Anti-Infective In Vitro Assay

The antibacterial potency of Compound 34 was measured using the in vitro broth microdilution assay under assay conditions described by the Clinical and Laboratory Standards Institute. In this assay, the Minimum Inhibitory Concentration (MIC) is defined as the lowest concentration of an agent that completely inhibits visible growth in vitro of the microorganism. The test substance was dissolved in 100% DMSO (unless stated below), suspended completely by sonication or vortexing, diluted by 2-fold serial titrations in the same vehicle, for a total of 10 test concentrations (final concentrations: 100, 50, 25, 12.5, 6.25, 3.125, 1.56, 0.78, 0.39, 0.2 μM). A 4 μL aliquot of each dilution was added to 196 μL of broth medium seeded with the organism suspension in wells of a 96 well plate (bacterial count: 2-8×10⁵ colony forming units/mL final). The final volume was 200 μL in each well and the final DMSO concentration was 2 percent. The incubation time is either 1 or 2 days at 36° C. All strains tested underwent aerobic respiration, except for ATCC BAA-1805 and ATCC 29399. Following incubation, the test plates were visually examined and wells were scored for growth or complete growth inhibition to define the minimum inhibitory concentration. Each test substance was evaluated in duplicate and the results below are the duplicate test values. Vehicle-control and an active reference agent were used as blank and positive controls, respectively. Results are shown in Table 5.

TABLE 5
Activity of Compound 34 against various ATCC strains

| | | Broth | MIC | Comparator | | |
|---|---|---|---|---|---|---|
| Strain | Organism | Medium | (μM) | Compound | MIC (μg/mL) | MIC (μM) |
| ATCC | Enterococcus | CAMHB II | 3.13 | Tigecycline | 0.063 | 0.11 |
| 51299 | faecalis | | | | | |
| ATCC | Enterococcus | CAMHB II | 1.56 | Tigecycline | 0.063 | 0.11 |
| BAA- | faecium | | | | | |
| 2320 | | | | | | |
| ATCC | Staphylococcus | CAMHB II | 3.13 | Vancomycin | 1.0 | 0.69 |
| BAA- | aureus | | | | | |
| 1717 | | | | | | |
| ATCC | Staphylococcus | CAMHB II | 3.13 | Vancomycin | 1.0 | 0.69 |
| 12228 | epidermidis | | | | | |
| ATCC | Staphylococcus | CAMHB II | 3.13 | Vancomycin | 1.0 | 0.69 |
| 19636 | aureus | | | | | |
| ATCC | Propionibacterium | RCM | 1.56 | Vancomycin | 0.5 | 0.35 |
| 29399 | acnes | | | | | |

### Example 4: Activity of Compound 34 in Anti-Infective In Vitro Assay (S. aureus)

The antibacterial potency of Compound 34 was measured using the in vitro broth microdilution assay under assay conditions described by the Clinical and Laboratory Standards Institute. In this assay, the Minimum Inhibitory Concentration (MIC) is defined as the lowest concentration of an agent that completely inhibits visible growth in vitro of the microorganism. The test substance was dissolved in 100% DMSO (unless stated below), suspended completely by sonication or vortexing, diluted by 2-fold serial titrations in the same vehicle, for a total of 11 test concentrations. A 4 μL aliquot of each dilution was added to 196 μL of broth medium seeded with the organism suspension in wells of a 96 well plate (bacterial count: 2-8×10⁵ colony forming units/mL final). The final volume was 200 μL in each well and the final DMSO concentration was 2%. The incubation time is 1 day at 36° C. All strains tested underwent aerobic respiration. Following incubation, the test plates were visually examined and wells were scored for growth or complete growth inhibition to define the minimum inhibitory concentration. Each test substance was evaluated in duplicate and the results below are the duplicate test values. Vehicle-control and an active reference agent were used as blank and positive controls, respectively. Results are shown in Table 6.

TABLE 6
Activity of Compound 34 against various S. aureus strains

| | Broth | MIC | MIC | Comparator | | |
|---|---|---|---|---|---|---|
| Strain | Medium | (μg/mL) | (μM) | Compound | MIC (μg/mL) | MIC (μM) |
| ECL 2963646 | CAMHB II | 2.0 | 2.37 | Linezolid | 2.0 | 5.93 |
| NARSA VRS1 | CAMHB II | 2.0 | 2.37 | Linezolid | 2.0 | 5.93 |
| NARSA VRS11b | CAMHB II | 1.0 | 1.18 | Linezolid | 2.0 | 5.93 |
| NARSA VRS2 | CAMHB II | 2.0 | 2.37 | Linezolid | 2.0 | 5.93 |
| NARSA VRS3a | CAMHB II | 2.0 | 2.37 | Linezolid | 4.0 | 11.86 |

### Example 5: Activity of Compound 34 Compared to Comparators

S. aureus (BAA-1556) was from ATCC. Mupirocin (Cat # M2955) was purchased from TCI chemicals (India) Pvt. Ltd., ciprofloxacin (Cat #17850) and linezolid (Cat #PZ0014) were purchased from Sigma-Aldrich Chemicals Pvt. Ltd, India, Cation adjusted Mueller Hinton broth (CAMHB) and Mueller Hinton Agar (MHA) (Cat # M1657) were from HiMedia Laboratories Pvt. Ltd, India.

The MIC assay was done based on CLSI guidelines in a microtitre plate format (1, 2). Preparation and dilution of Test Item (TI) stock solutions: A 1280 μg/ml stock of Compound 34 was prepared by dissolving 1.28 mg in 1.0 ml of sterile water to obtain the Master Stock (MS) solution. The MS solution was diluted 2-fold to obtain a series of Working Stock (WS) solutions (Table 7). The details of the final concentrations of the test items assayed are shown in Table 1. The final concentration of solvent in the assay was 2.5% and the assay volume was 100 al.

TABLE 7
Preparation of WS solutions and final assay concentrations for MIC

| | WS Conc. | Volume WS | Vol of | Vol of | Final Assay | Final compound |
|---|---|---|---|---|---|---|
| No | (μg/ml) | in well (μl) | CMHB (μl) | Culture (μl) | Vol. (μl) | conc. (μg/ml) |
| 1 | 640 | 2.5 | 47.5 | 50 | 100 | 16 |
| 2 | 320 | 2.5 | 47.5 | 50 | 100 | 8 |
| 3 | 160 | 2.5 | 47.5 | 50 | 100 | 4 |
| 4 | 80 | 2.5 | 47.5 | 50 | 100 | 2 |
| 5 | 40 | 2.5 | 47.5 | 50 | 100 | 1 |
| 6 | 20 | 2.5 | 47.5 | 50 | 100 | 0.5 |
| 7 | 10 | 2.5 | 47.5 | 50 | 100 | 0.25 |
| 8 | 5 | 2.5 | 47.5 | 50 | 100 | 0.125 |
| 9 | 2.5 | 2.5 | 47.5 | 50 | 100 | 0.0625 |
| 10 | 1.25 | 2.5 | 47.5 | 50 | 100 | 0.03125 |
| 11 | 0.625 | 2.5 | 47.5 | 50 | 100 | 0.015625 |

Preparing the inoculum: One day before initiation of experiment, an isolated colony of S. aureus (ATCC BAA-1556) was inoculated into sterile CAMHB and incubated at 37° C. to exponential phase. After incubation, the turbidity was adjusted to that of a 0.5 McFarland Standard (approximately equivalent to 1.0×10⁸ CFU/ml) and diluted to ˜1.0×10⁶ CFU/ml.

MIC Assay: The MIC assay was performed in a 96 well microtitre plate in a total assay volume of 100 μl (Table 7). Each well containing different concentrations of test compound (concentration range between 0.015625 and 16.0 μg/ml) was inoculated with 50 μl of bacterial suspension (prepared from the inoculum grown overnight and adjusted approximately to 5.0×10⁵ CFU/ml) along with culture control (CC, culture in broth), broth control (BC, broth only), and vehicle control (VC, 2.5% solvent in broth plus culture). The plate was incubated at 37° C. for 24 hr and turbidity was measured spectrophotometrically (Absorbance microplate reader, BioTek® India, ELx800™) at 600 nm and visually. The experiment was done in duplicates. The inoculum was also plated for enumeration of bacteria. The MIC was defined as the lowest concentration of test compound that prevented bacterial growth (lack of turbidity by OD at 600 nm and visual inspection relative to no growth control). The results are shown in Table 8.

TABLE 8
Mean MIC of test and reference compounds
on S. aureus (ATCC BAA-1556)

| Compound | MIC [μg/ml] | MIC μM |
|---|---|---|
| Compound 34 | 2.00 | 2.37 |
| Mupirocin | >16 | >31.96 |
| Ciprofloxacin | 2.00 | 6.04 |
| Linezolid | 1.00 | 2.96 |

The claims defining the invention:

1. A compound of formula (I): 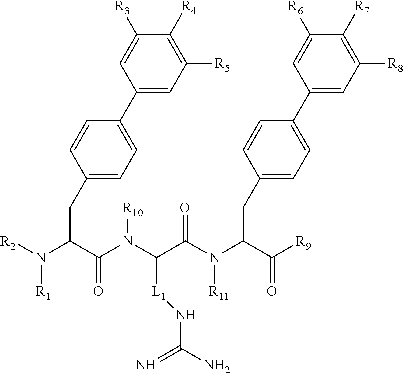
or a pharmaceutically acceptable salt or solvate thereof;
wherein L₁ is —(CH₂)ₘ—, C₂-C₅ alkenylene, C₂-C₅ alkynylene, C₃-C₆ cycloalkylene, phenylene or benzylene, wherein m is selected from 1 to 5;
wherein R₁ and R₂ are independently selected from H or CH₃, or
R₁ is selected from H or CH₃ and R₂ is 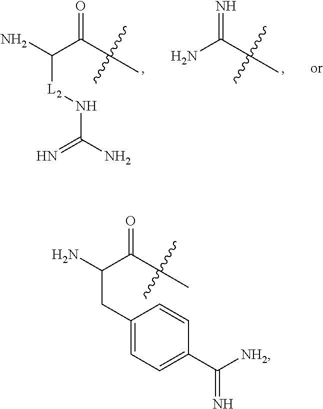
wherein L₂ is —(CH₂)ₙ—, C₂-C₅ alkenylene, C₂-C₅ alkynylene, C₃-C₆ cycloalkylene, phenylene or benzylene, wherein n is selected from 1 to 5;
wherein R₃-R₈ are independently H, Cl, I, Br or F;
wherein at least one of R₃-R₈ is Cl, I, Br or F;
wherein R₉ is 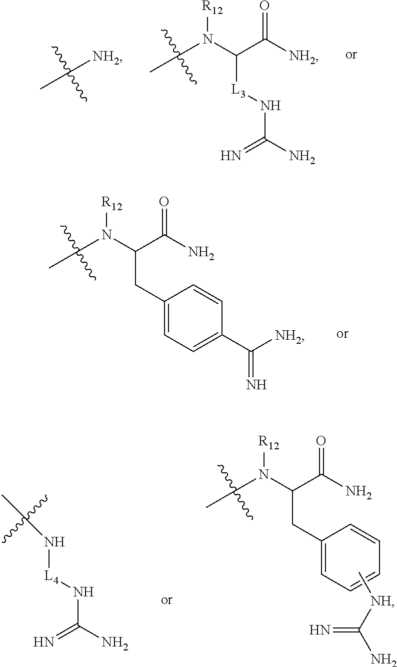
wherein L₃ is —(CH₂)ₒ—, C₂-C₅ alkenylene, C₂-C₅ alkynylene, C₃-C₆ cycloalkylene, phenylene or benzylene, wherein o is selected from 1 to 5;
wherein L₄ is —(CH₂)ₚ—, C₂-C₅ alkenylene, C₂-C₅ alkynylene, C₃-C₆ cycloalkylene, phenylene or benzylene, wherein p is selected from 1 to 5;
wherein R₁₀, R₁₁ and R₁₂ are independently H or —CH₃.

2. A process of making compounds of formula (I) 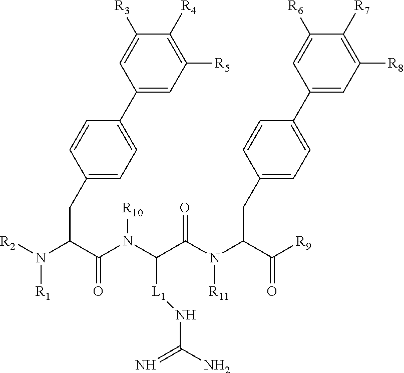
or a pharmaceutically acceptable salt or solvate thereof;
wherein L₁ is —(CH₂)ₘ—, C₂-C₅ alkenylene, C₂-C₅ alkynylene, C₃-C₆ cycloalkylene, phenylene or benzylene, wherein m is selected from 1 to 5;
wherein R₁ and R₂ are independently selected from H or CH₃, or
R₁ is selected from H or CH₃ and R₂ is 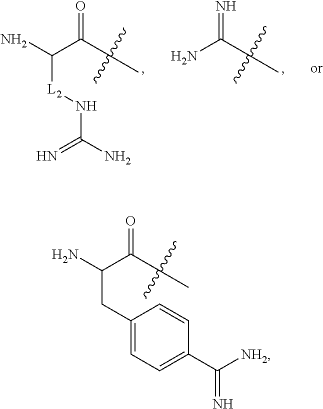
wherein L₂ is —(CH₂)ₙ—, C₂-C₅ alkenylene, C₂-C₅ alkynylene, C₃-C₆ cycloalkylene, phenylene or benzylene, wherein n is selected from 1 to 5;
wherein R₃-R₈ are independently H, Cl, I, Br or F;
wherein at least one of R₃-R₈ is Cl, I, Br or F;
wherein R₉ is 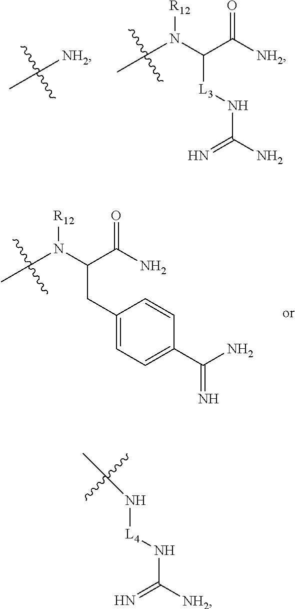
wherein L₃ is —(CH₂)ₒ—, C₂-C₅ alkenylene, C₂-C₅ alkynylene, C₃-C₆ cycloalkylene, phenylene or benzylene, wherein o is selected from 1 to 5;
wherein L₄ is —(CH₂)ₚ—, C₂-C₅ alkenylene, C₂-C₅ alkynylene, C₃-C₆ cycloalkylene, phenylene or benzylene, wherein p is selected from 1 to 5;
wherein R₁₀, R₁₁ and R₁₂ are independently H or —CH₃,
by reacting following compounds of formula (II): 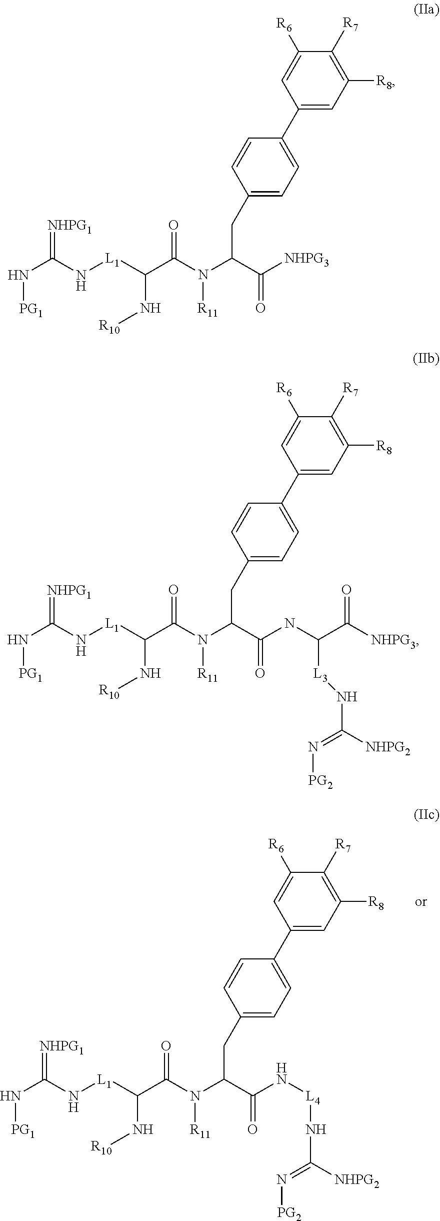 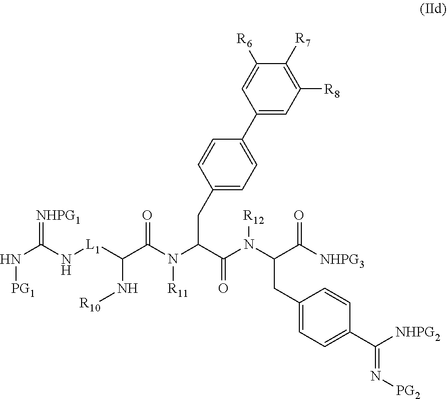
wherein R₆, R₇, R₈, R₁₀, R₁₁, R₁₂, L₁, L₃ and L₄ have the meaning given above; wherein PG₁, PG₂ and PG₃ can be any protecting group such as t-Boc or Pbf;
in the presence of an amide/peptide coupling reagent with compounds of the formula (III) 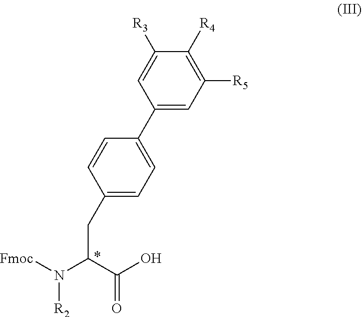
wherein R₂-R₅ are defined as mentioned above,
wherein * denotes an stereogenic carbon;
and deprotection with an acid.

3. A compound of formula (I) according to claim 1 or a pharmaceutically acceptable salt or solvate therefore for use as a medicament.

4. A compound of formula (I) according to claim 1 or a pharmaceutically acceptable salt or solvate thereof for use in the treatment of diseases, disorders and conditions which are a bacteria infection.

5. The compound of claim 4 wherein the bacterial infection is caused by a Gram-positive bacteria strain.

6. The compound of claim 4 wherein the bacterial infection is caused by Staphylococcus aureus, Methicillin-resistant Staphylococcus aureus, Streptococcus pyogenes, Streptococcus pneumoniae or Propionibacterium acnes.

7. The compound of claim 4 wherein the bacterial infection is caused by bacteria that have gained a resistance against the penicillin-type antibiotics, Vancomycin, Linezolid, Retapamulin, Tigecycline or Mupirocin.

8. The compound of claim 4 wherein the bacterial infection is treated by topical use of the compound.

9. Use of a compound of formula (I) according to claim 1 or a pharmaceutically acceptable salt or solvate thereof in the manufacture of a medicament for the treatment of a disease, disorder or condition selected from any bacterial infection.

10. The compound of claim 9 wherein the bacterial infection is caused by a Gram-positive bacteria strain.

11. The use according to claim 9 wherein the bacterial infection is caused by Staphylococcus aureus, Meticillin-resistant Staphylococcus aureus, Streptococcus pyogenes, Streptococcus pneumoniae or Propionibacterium acnes.

12. The use according to claim 9 wherein the bacterial infection is caused by bacteria that have gained a resistance against the penicillin-type antibiotics, Vancomycin, Linezolid, Retapamulin, Tigecycline or Mupirocin.

13. The use according to claim 9 wherein the bacterial infection is treated by topical application of a compound of formula (I).

14. A method of treating a bacterial infection caused disease, disorder or condition in a subject in need of such treatment, comprising administered to said subject a compound of formula (I) according to claim 1 or a pharmaceutically acceptable salt or solvate thereof.

15. The method according to claim 14 wherein the bacterial infection is treated by topical application of a compound of formula (I).

16. A method of treating or preventing an acne condition in a subject in need of such treatment, comprising administering to said subject a compound of the formula (I) or a pharmaceutically acceptable salt or solvate thereof.

17. A method of disinfecting a surface against bacterial contamination, comprising applying to said surface a compound of the formula (I) or an acceptable salt or solvates thereof.

18. A pharmaceutical composition comprising a compound of formula (I) according to claim 1 or a pharmaceutically acceptable salt or solvate thereof and a pharmaceutically acceptable excipient.

19. A pharmaceutical composition comprising a compound of formula (I) according to claim 1 or a pharmaceutically acceptable salt or solvate thereof and a pharmaceutically acceptable excipient for use in the treatment of a bacterial infection.
